(19) United States
(12) Patent Application Publication
(10) Pub. No.: US 2013/0210646 A1
(43) Pub. Date: Aug. 15, 2013

(54) HEMOMOSAIC: HIGH-THROUGHPUT TECHNIQUE FOR RARE CELL DETECTION IN LIQUID SAMPLES BY MASSIVELY MULTIPLEXED PCR IN A PHOTOLITHOGRAPHIC MATRIX

(71) Applicants: University of Southern California, (US); California Institute of Technology, (US)

(72) Inventors: Emil P. Kartalov, Pasadena, CA (US); Michael Kahn, Altadena, CA (US); Axel Scherer, Barnard, VT (US)

(73) Assignees: California Institute of Technology, Pasadena, CA (US); University of Southern California, Los Angeles, CA (US)

(21) Appl. No.: 13/763,536

(22) Filed: Feb. 8, 2013

Related U.S. Application Data
(60) Provisional application No. 61/597,078, filed on Feb. 9, 2012.

(51) Int. Cl. C12Q 1/68; C12M 1/12; C12Q 1/24

(52) U.S. Cl. CPC C12Q 1/686; C12Q 1/24; C12M 25/00
USPC 506/7; 506/23; 506/40

(57) ABSTRACT

Described microfluidic technology focused on: 1) direct integration of microfluidic devices with solidified liquid tissue samples, 2) subordination of architectural and operational principles of microfluidic devices specific tissue structure and needs and/or 3) on-chip sample acquisition integrated with the detection measurement within the same device. In contrast to conventional methods of off-chip sample prep and subsequent insertion into a detection device, new applications are possible on solidified liquid or solid tissue samples, such as in situ PCR.

### CROSS-REFERENCE TO RELATED APPLICATIONS

This application includes a claim of priority under 35 U.S.C. §119(e) to U.S. provisional patent application No. 61/597,078, filed Feb. 9, 2012.

### GOVERNMENT LICENSE RIGHTS

The U.S. Government has a paid-up license in this invention and the right in limited circumstances to require the patent owner to license others on reasonable terms as provided for by the terms of Grant No. R00 awarded by the National Institutes of Health.

### BACKGROUND

Metastatic processes in cancer development are an emerging focus of research considering that 90% of cancer patients do not die because of their primary tumor, they are killed by metastatic disease. Further understanding of metastatic processes is clearly an essential point in tackling cancer disease. Although most data on the prognostic value of disseminated tumor cells (DTCs) are for breast cancer, several single institutional studies that include patients with colon, lung and prostate cancer associate the presence of DTCs at primary surgery with subsequent metastatic relapse.

Ultrasensitive methods have been recently developed to detect circulating tumor cells (CTCs) in the peripheral blood and disseminated tumor cells (DTCs) in the bone marrow (BM) of cancer patients. Studies with these new methods indicate that BM is a common homing organ and a reservoir for DTCs derived from various organ sites including breast, prostate, lung and colon. Peripheral blood analyses, however, are significantly more convenient for patients than invasive BM sampling and many research groups are currently assessing the clinical utility of CTCs for prognosis and monitoring response to systemic therapies.

The detection of CTCs in the peripheral blood of cancer patients holds great promise but remains technically challenging. However, if a relatively simple, cost effective and reliable assay for detection of CTCs in peripheral blood can be developed, there are a number of important clinical applications. CTC/DTC analyses could be evaluated in the context of predicting the prognosis of cancer patients, selecting the most efficient therapy and monitoring these therapies by repeated blood analyses. The molecular profiling of CTCs/DTCs could significantly impact prognosis and benefit from therapy, as well as be utilized in the context of new therapeutic agents to determine efficacy. Current methods of detecting these cells by size-membrane filtration, density, the expression of cell surface markers (EpCAM, for positive- and CD45 for negative selection; anti-EpCAM or anti-CD45 antibodies conjugated with magnetic beads are used to enrich CTCs in a magnetic field) and/or invasive capacity (adherence and invasion of fluorescent matrix) each possess significant limitations in failing to anticipate transient biochemical transition related to epithelial-to-mesenchymal transition (EMT), a process attributed to disseminating cancer cells, or by lacking sensitivity for detection of these rare populations. Therefore, there is a great need in the art for novel and inventive approaches to detect CTCs in peripheral blood samples in an unbiased fashion would have significant practical value.

### BRIEF DESCRIPTION OF THE DRAWINGS

FIG. 1 depicts photolithographic masking of apparatus. In a top view, a photocurable substance (gray) is exposed to UV light through a (A) photolithographic mask bearing a hexagonal matrix pattern. The dark areas in the mask absorb the UV and thus protect the photoresist under them, while the light areas transmit the UV light to the underlying photoresist. (B) the result is a matrix of hexagonal access wells on top of the tissue. Based on the size of the access wells, as few as one cell (blue circle) per well can be isolated (C) defined access wells can then be filled with PCR reagents, and positively detected (green) in the presence of a cell of interest.

FIG. 2 depicts photolithographic masking of apparatus. In a side view, (A) cellular material, such as solidified liquid tissue (pink) is deposited on a support (green) photocurable substance, gray) (B) which is then exposed to UV light (purple) through a photolithographic mask containing blocking (black) and non-blocking (white) regions, the UV light able to pass through non-blocking reigons. The result is (C) defined access wells, which can be (D) filled at the same time with the same PCR reagents. (E) the matrix is sealed with another glass slide (green) coated with cured elastomer (red) to serve as a gasket layer. The assembled chip is then processed in a standard flat-top PCR machine. (F) If a cell is segregated in the access well, PCR reaction for analytes of interest would positively detect (green star) as shown in the representation.

FIG. 3 depicts a cross-sectional view of a series of steps in which tissue masking selectively destroys DNA.

FIG. 4 depicts a cross-sectional view of a series of steps where tissue isolation or masking is performed through lamination with a photosensitive material.

FIG. 5 depicts a top view of an embodiment in which tissue isolation targets multiple areas of interest occurring by lamination with a photosensitive material.

FIG. 6 depicts a cross sectional view where a tissue is integrated with microfluidic elements.

FIG. 7 depicts an embodiment (A) related to FIG. 5, and illustrates a top view of a customized chambered microfluidic device. (B) is related to FIG. 5, and illustrates a top view of a standardized chambered microfluidic device.

FIG. 8 depicts a cross-sectional view of an embodiment where tissue encapsulation is performed.

FIG. 9 depicts an embodiment in which a maskless chambered microfluidic device encapsulates the tissue encapsulates a tissue without customized photomasking while specificity of micro-isolation is achieved through active control of arrays of valves allowing specificity of microisolation to be achieved through active control of arrays of valves.

FIG. 10 depicts a top view of a matrix of microfluidic wells.

FIG. 11 depicts a series of steps for the parallel processing of isolated tissue subsections.

FIG. 12 depicts a series of steps for the parallel processing of isolated tissue subsections that allows step-wise administration of biochemical agents.

FIG. 13 depicts an optical setup for dynamic optical array masking.

FIG. 14 depicts (A) an access well on tissue in overhead light (B) in fluorescence image.

FIG. 15 depicts results demonstrating successful in-situ PCR with colorectcal (CRC) tissue.

FIG. 16 depicts a matrix of smallest wells on tissue.

FIG. 17 depicts computer assisted design (CAD) of circuit board printing techniques showing (A) top-down view (B) three-quarter view (C) side width profile and (D) additional side longitiduinal profile.

FIG. 18 depicts final assembly of apparatus embodiment in (A) three-quarter view (B) and assembly of device.

### DETAILED DESCRIPTION OF THE INVENTION

All references herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Unless defined otherwise, technical and scientific terms used herein have the same meaning as commonly understood by one of ordinary skill in the art to which this invention belongs. Singleton et al., Dictionary of Microbiology and Molecular Biology 3.sup.rd ed., J. Wiley & Sons (New York, N.Y. 2001); March, Advanced Organic Chemistry Reactions, Mechanisms and Structure 5.sup.th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Blocking region” refers to a region of a blocking support that functions to block photons (e.g. non-transparent region blocking photons by absorption). In an embodiment, a blocked region can be a region of chrome covering the blocking support. One skilled in the art would appreciate that many metals or other materials could be used, for example, to block photon exposure. For example, titanium can be deposited on platinum, covered with a photosensitive material, and then similarly oxidized to produce a chemically and mechanically resistant oxide that can serve as a photon shield for agents damaging to micro-components such as cellular material, such as UV exposure. In some embodiments, a material forming a blocking region can also further be a polarizer as will be understood by a skilled person.

“Blocking support” as used herein refers to a support that can be used to support a blocking region. For example, a blocking support can be transparent, or in general configured for allowing passage of a desired lighting in one or more areas where there is no blocking region. “Cellular material” refers to biological material pertaining to a biological cell. As used herein, it can refer to sub-components of a biological cell, a single intact biological cell, a group of biological cells, or a tissue, such as liquid tissue.

“Digital image” refers to an image generated by a computer or other suitable electronic device. In an embodiment, a digital image can be provided, for example, by a set of instructions in software on a computer controlling the optical hardware. In an embodiment, the image can be a 2-D image.

“Illumination” refers to the exposure of light. Light can be visible or non-visible light, and can be one or more of UV, two-photon, or multi-photon light or additional examples of light of various wavelength suitable to be used in connection with biological components which are identifiable by a skilled person upon reading of the present disclosure

“Hardwired” refers to devices, methods and systems herein described or portions thereof, that are tailored for a specific biological component of interest. For example, a particular device is hardwired if it is configured to be suitable for a specific component, e.g. a specific tissue to be investigated using methods and systems herein described. In some embodiments, hardwired devices, methods and systems are herein described that are tailored not only for a specific biological component of interest, but also for a specific investigative approach of interest. For example, in some embodiments, hardwired masks are described that allow UV light to be directed only to areas of no interest so that the DNA, protein, or other biological material in those areas is damaged to various extents and even destroyed by photodamage. In some of those embodiments, damage in areas of interest not exposed to UV is minimized to various extents and in some cases even remain intact. The term “destroy” as used in the present disclosure with reference to an item indicates a damage level able to impact at least one biological activity associated to the item. The term “intact” as used in the present disclosure with reference to an item indicates a molecule that preserve all the biological activities associated to the item.

“Microfluidic” refers to a system or device for handling, processing, ejecting and/or analyzing a fluid sample including at least one channel having microscale dimensions. Microfluidic tissue isolation can be customized morphologically, functionally, and a combination of the two.

“Micro-isolation” refers to the isolation of micro-scale components (components having a size or measure in the order of micrometers) and in particular of light sensitive microscale components, which includes, but is not necessarily limited to, one or more biological components. A biological component refers to any organized substance forming part of a living matter, e.g. a cell, cellular material, membranes, organelles, proteins, nucleic acids and/or living organisms of any dimensions or a part thereof (e.g. tissues or various cell extracts). Micro-isolation as used herein can refer to the isolation of a single nucleus, a single cell or a biological component thereof, a group of individual cells, or a cluster of cells, or a group of clusters of cells, or a specific region of a tissue or a portion thereof, or even cellular organelles (e.g. cell nuclei). In particular, in some embodiments, methods and system herein described allow one to simultaneously address a distributed group of regions of interest across a tissue slide while each region can be a single nucleus, a single cell, a cluster of cells or a biological component thereof “Micro-isolation apparatus” refers to a device that aids in the micro-isolation of a microscale component (e.g. a biological component, which relates to biology, life and/or living processes, such as a cellular material).

“Photomask” as used herein refers to the blocking support comprising a blocking region and a light accessible region. The term “blocking” refers to the ability of an item to hinder the passage of light through the item. The term “light accessible” as used herein refers to the ability of an item to allow passage of light through the item. In some embodiments, the photomask can be any type of transparent support (light accessible region) having a non-transparent region (blocking region). In some embodiments, the transparent support can further be at least in part, semi-transparent, or translucent and/or include different blocking portions with different blocking and light accessible capabilities (e.g. limited to one or more selected wavelengths for one or more areas of the photomask). In some embodiments, the photomask can be a physical object (e.g. a glass slide partially covered with chrome, or a transparency partially covered with ink or other blocking material). In some embodiments, the photomask can be purely or partially digital. For example, in some embodiments, the photomask can include a series of instructions to a micro-minor array, which operates so that some minor elements are activated while others are not. In some of those embodiments, they activate minor elements to form a photomask pattern on a sample with respect to an illumination light reflected onto the sample by the micro-mirror array. Additional embodiments are encompassed by the present disclosure wherein a photomask is dynamic photomask, as the instructions are dynamically defined in addition or in the alternative to photomask wherein physical blocking material (e.g. chrome coating) blocks light on a suitable support (e.g. glass slide).

“Region of interest” as used herein pertains to a targeted area within cellular material. Definition of a targeted area can be of any dimensions and include one or more cellular material depending on the experimental design of choice. For example, the region of interest can be an area that is sought to be preserved, or an area that is sought to be damaged or even destroyed. In a further example, the region of interest can be as small as a DNA molecule, or as large as an entire tissue sample, a group of topologically non-contiguous targeted areas in the tissue sample, which are all to be isolated and/or extracted at a same or a different time.

“Support” as used herein refers to any type of support in which cellular material can be mounted. One type of support is a glass slide, although one skilled in the art would recognize that many materials can provide support for cellular material.

As described, the detection of CTCs in the peripheral blood of cancer patients holds great promise but remains technically challenging. If a relatively simple, cost effective and reliable assay for detection of CTCs in peripheral blood can be developed, there are a number of important clinical applications. CTC/DTC analyses could be evaluated in the context of predicting the prognosis of cancer patients, selecting the most efficient therapy and monitoring these therapies by repeated blood analyses. The molecular profiling of CTCs/DTCs could significantly impact prognosis and benefit from therapy, as well as be utilized in the context of new therapeutic agents to determine efficacy. In particular, agents that target stem/progenitor cells, where the effects on the bulk tumor may be minimal would benefit significantly from methodologies to profile CTC/DTC. Finally, CTC/DTC analysis will contribute to a better understanding of the complex metastatic process in cancer patients, which might unravel new strategies to eradicate metastatic cells or control their outgrowth into life-threatening overt metastases.

To date, enrichment of CTCs from the peripheral blood of cancer patients are based on four principles: size (membrane filter devices), density (Ficoll centrifugation), the expression of cell surface markers (EpCAM, for positive- and CD45 for negative selection; anti-EpCAM or anti-CD45 antibodies conjugated with magnetic beads are used to enrich CTCs in a magnetic field) and invasive capacity (adherence and invasion of fluorescent matrix).

Positive selection is usually carried out with antibodies against the epithelial cell adhesion molecule (EpCAM) and subsequent immunocytological detection of CTCs is performed with antibodies to cytokeratins (CKs), the intermediate filaments of epithelial cells. Among the current EpCAM/CK-based technologies, the FDAapproved CellSearch™ system has gained considerable attention over the past six years. In parallel, a microfluidic platform called CTC-chip, which consists of an array of anti-EpCAM antibody-coated microposts, was presented and applied to the analysis of blood samples from patients with solid tumors. The high CTC counts in cancer patients and the frequent detection of positive events in healthy controls warrants further investigations on the specificity of this interesting new assay.

The current gold standard for imaging CTCs is fiber-optic array scanning technology (FAST) cytometers, which uses laser scanning to locate rare cells almost 1000 times faster than digital microscopy. With this high scan rate, no enrichment of CTCs is required. The procedure begins with conventional staining of rare cells with fluorescent probes. The probes are attached to the cell through an antibody reaction that is specific to the phenotype of the cell. The peripheral blood cells are then rapidly scanned for the presence of these probes on CTCs using a directed laser.

Importantly, however all EpCAM-based enrichment systems share the same limitation: EpCAM can be down-regulated during epithelial-to-mesenchymal transition (EMT), a process attributed to disseminating cancer cells. Recent research indicates that this transition might affect tumor cells with stem cell-like properties in particular. Assays targeting specific mRNAs are the most widely used alternative to immunological assays to identify CTCs. Many transcripts (e.g. encoding CK18, CK19, CK20, Mucin-1, Prostate specific antigen and Carcino-embryonic antigen), however, are also expressed at low levels in normal blood and BM cells so quantitative RT-PCR assays with validated cutoff values are required to overcome this problem. Moreover, gene transcription might be down-regulated in CTCs and DTCs (e.g. in the course of EMT), which argues in favor of multimarker RT-PCR approaches.

Therefore, novel and inventive approaches to detect CTCs in peripheral blood samples in an unbiased fashion would have significant practical value.

Described herein is a paradigm shift in microfluidic technology focused on: 1) direct integration of microfluidic devices with solidified liquid tissue samples, in contrast to conventional methods of off-chip sample extraction followed by sample insertion in microfluidic devices, 2) subordination of architectural and operational principles of microfluidic devices specific tissue structure and needs, in contrast to certain conventional method of building devices according to fluidic function alone and without regard to tissue structure, and/or 3) on-chip sample acquisition integrated with the detection measurement within the same device, including application of in situ PCR, which is not possible with conventional methods of off-chip sample prep and subsequent insertion into a detection device.

Described herein is an apparatus, including a support, cellular material mounted upon the support, photomask including at least one non-blocking region and at least one blocking region, wherein the photomask is placed over the cellular material such that each of the at least one blocking region is positioned to correspond to a region of interest of the cellular material. In a different embodiment, the support includes a glass slide, a quartz slide or a transparent polymer slide. In a different embodiment, the at least one blocking region is a metal. In a different embodiment, the at least one blocking region is a polarizer. In a different embodiment, the at least one blocking region is capable of limiting photon exposure. In a different embodiment, the photons are derived from one or more of X-ray, UV, two-photon, or multi-photon illumination light. In a different embodiment, the at least one non-blocking region is configured to allow passage of photons.

In a different embodiment, the chambered microfluidic device includes photosensitive material located above the cellular material, and wherein the photosensitive material includes a plurality of access wells corresponding to a plurality of respective areas of interest of the cellular material. In a different embodiment, the plurality of access wells are interconnected through a series of channels in the microfluidic device. In a different embodiment, the chambered microfluidic device is configured for a coverage of 50%, 55%, 60%, 65%, 70%, 75%, 80%, 81%, 82%, 83%, 84%, 85%, 86%, 87%, 88%, 89%, 90% or more. Coverage, C, as a geometric calculation shows that the coverage C=1/{1−T/[R*sqrt(3)]}, where T is the wall thickness and R is the radius of an example hexagon. For 20 μm-wide wells separated by 10 μm walls, C=63%. If the wells are the same but the walls are 2 μm-wide, C=90%.

In various embodiments of using photolithographically defined masks where the biological material includes cells and experimental designs directed to isolate and/or analyze individual cells, a dense porous “honeycomb” arrangement of predetermined access wells can be used in a device, methods or systems herein described such that the pre-existing access wells can be placed accordingly over cellular regions according to the specific analysis of choice (e.g. to perform protein and/or DNA analysis of regions of interest selected).

In some embodiments, the configuration of a matrix of access wells herein described is not limited to desirable areas alone. In some of those embodiments, some or all wells can be analyzed simultaneously but separately, so that no predetermined regions are necessary. Accordingly, in an embodiment, of devices methods and systems herein described a photomask can be designed to single out only the areas of interest and/or to include a repeating regular or irregular geometric pattern of choice (e.g. circles, squares, or hexagons in rectangular, checkered, or honeycomb formation) of appropriately chosen size and spacing, e.g. to contain only one microscale component or portion thereof (e.g. one cell per well).

In several embodiments, wells of a masked or maskless matrix of access can include one or more reaction mixtures. A reaction mixture can be any mixture containing components necessary for a biochemical reaction to occur. Reaction mixtures can include, but are not limited to, components necessary for PCR, real-time PCR, RT-PCR, flow cytometry, fluorescent labeling, FRET, DNA sequencing, protein-protein interaction assays, immunoassays, protein-nucleic acid assays, and any other biological reaction known in the art.

A matrix of microfluidic wells allows incomparable parallelism in extracting the sequencing information while preserving the morphological and contextual information from the solidified liquid tissue sample. As an illustrative example, while present methods can provide for 96, 384, or 1536 simultaneous parallel reactions, various embodiments of the present invention can performed over 1536 simultaneous parallel reactions, such as 2000, 3000, 4000, 5000, 6000, 7000, 8000, 9000, 10,000 or more parallel reactions. This enables large-scale mapping of two-dimensional spatial distribution of mutations across a tumor section. In many embodiments, the optimal well size is the same size as a mammalian cell (approximately 20 μm), although one skilled in the art will recognize that different well sizes can be used for different applications, this includes, for example 1, 2, 3, 4, 5, 6, 7, 8, 9, 10, 11, 12, 13, 14, 15, 16, 17, 18, 19, 20, 30, 40, 50, 60, 70, 80, 90, 100 or more μm sized wells. For example, it is understood that all of the various embodiments further described herein, the integration of microfluidic techniques and micro-isolation of cellular material molds the microfluidic architectures in accordance with the particular structure of each specific biological component to be isolated. In particular, in some of those embodiments, the approach described herein is mainly built around the cellular material, following the solidified liquid tissue structure such that the devices and methods described herein are adapted to the specific geometry of particular solidified liquid tissues or other biological components. By contrast, in some of the traditional approaches microfluidic devices are structured taking only in accordance with engineering considerations (e.g. path minimization, fluidic efficiency), while the biological components in applied devices are forced to comply with these engineering presets and are not taken into consideration.

In particular, the massively parallel technique achieves digital noise reduction in liquid tissues for detection of rare cells without bias caused by sample manipulation or preparation. A particular example includes the successful application of in situ PCR. Further, there is no signal loss, which inevitably causes poor sensitivity for detection of rare cells. A further advantage is the application of multiplexing biomarker detection to eliminate detection techniques relying upon the appearance of sometimes transient biomarkers, such as epithelial-mesenchymal transition (e.g., EpCAM).

In another aspect, further described herein is a method for selectively isolating cellular material, including positioning cellular material on a support, and depositing a photosensitive material on the cellular material, applying a photomask including at least one non-blocking region and at least one blocking region onto the photosensitive material, and exposing photons to the photosensitive material through the at least one non-blocking region in order to define at least one access well, and wherein each of the at least one blocking region corresponds to a region of interest of the cellular material. In a different embodiment, the photons are generated by arrays of micro- and nano-lasers light-emitting diodes, or photonic crystal devices, and/or reflected onto the sample by micro-mirror arrays.

In another aspect, also described herein is a method to detect analytes, including solidifying liquid tissue, positioning the solidified liquid tissue on a support, depositing a photosensitive material on the solidified liquid tissue, applying a photomask including at least one non-blocking region and at least one blocking region onto the photosensitive material, exposing photons to the photosensitive material through the at least one non-blocking region in order to define at least one access well, and wherein each of the at least one blocking region corresponds to a region of interest of the solidified liquid tissue, filling the at least one access well with a reaction mixture including agents and components necessary for reaction to detect analytes; and performing the reaction simultaneously in the at least one access well, thereby detecting the analytes.

In a different embodiment, the reaction includes one or more reagents for PCR, real-time PCR, RT-PCR, flow cytometry, fluorescent labeling, FRET, DNA sequencing, protein-protein interaction assays, immunoassays, protein-nucleic acid assays. In a certain embodiment, the PCR is PCR is in situ PCR not requiring “lossy” sample manipulation (e.g., extraction or purification) prior to detection of analytes in the sample. In a different embodiment, the signal detection is accomplished by scanning a completed reaction using a fluorescence scanner and/or a fluorescence microscope. In various embodiments, the solidified tissue and coverage of the at least one access well are each configured for detection of 200, 100, 90, 80, 70, 60, 50, 40, 30, 20 or fewer cells per access well. In a different embodiment, the solidified tissue and coverage of the at least one access well are each configured for detection of 10, 5, 2 or fewer cells per access well. In a different embodiment, the at least one access well are substantially hexagonal. In various embodiments, the at least one access well can be square, rectangular, trapezoidal, or any other shape that can be configured using a photolithographic mask. In a different embodiment, performing the reaction simultaneously in the at least one access well is massively parallel. This includes, for example, 2000, 3000, 4000, 5000, 6000, 7000, 8000, 9000, 10,000 or more parallel reactions. In a different embodiment, the solidified tissue and coverage of the at least one access well are each configured for detection of 10, 5, 2 or fewer cells per access well. In various embodiments, the liquid tissue is blood, cerebrospinal fluid, bone marrow, lymph fluid, interstitial fluid, plasma, or similar liquid tissues known to one of ordinary skill. In various embodiments, solidifying liquid tissue may be achieved by ordinary drying, adding to a solidifying agent such as agarose or gelatin, or various polymers know to one of ordinary skill. As used throughout the description, solid, or solidified in various embodiments include both solid and semi-solid forms of liquid. In particular, in certain embodiments, microfluidics is the micro-manipulation of fluids, and can be integrated with biochemical applications for microscale analyses of cells with further implementation identifiable by a skilled person.

In another aspect, further described herein is a micro-isolation apparatus including a support, a cellular material mounted upon the support, a photomask including a transparent region and a non-transparent blocking region, the non-transparent blocking region covering at least a portion of the transparent region, and wherein the photomask is placed over the cellular material such that the blocking region is positioned to correspond to a region of interest of the cellular material to minimize damage to the cellular material in the region of interest by illumination. In a different embodiment, the support and/or transparent region are a glass slide, a quartz slide or a transparent polymer slide. In a different embodiment, the non-transparent blocking region is a metal. In a different embodiment, the non-transparent blocking region is a polarizer. In a different embodiment, the non-transparent blocking region is capable of blocking photons. In a different embodiment, the photons are derived from one or more of X-ray, UV, two-photon, or multi-photon illumination light. In a different embodiment, the transparent region is configured to allow passage of a light sufficient to damage the cellular material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a micro-isolation apparatus including a support, a cellular material mounted upon the support, and a photosensitive material deposited on the cellular material, wherein the photosensitive material includes an access well positioned to correspond to a region of interest of the cellular material. In a different embodiment, the apparatus further including a chambered microfluidic device including at least one channel that provides access to the access well and is positioned adjacent to a photosensitive material located above the cellular material. In a different embodiment, the photosensitive material includes a plurality of access wells corresponding to a plurality of respective areas of interest of the cellular material. In a different embodiment, the plurality of access wells are interconnected through a series of channels in the microfluidic device. In a different embodiment, the series of channels are connected to one or more inputs and one or more outputs. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is micro-isolation apparatus including a first photosensitive material deposited on cellular material, adapted to be contained in the apparatus, wherein the first photosensitive material includes a first access well positioned to correspond to a region of interest of the cellular material, a first chambered microfluidic device including at least one channel that provides access to the first access well and is positioned adjacent to the first photosensitive material, a second photosensitive material adapted to be deposited on the cellular material opposite to the first photosensitive material, wherein the second photosensitive material includes a second access well positioned to correspond to an opposing side of the region of interest of the cellular material, and a second chambered microfluidic device including at least one channel that provides access to the second access well and is positioned adjacent to the second photosensitive material. In a different embodiment, the channel or channels of the first chambered microfluidic device provide an input to the region of interest of the tissue and/or cells and the channel or channels of the second chambered microfluidic device provide an output from the region of interest of the tissue and/or cells. In a different embodiment, the photosensitive material includes a plurality of access wells corresponding to a plurality of respective areas of interest. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a maskless micro-isolation apparatus including cellular material, a first chambered microfluidic device including multiple channels, wherein each channel provides access to respective regions of interest of the cellular material, and a second chambered microfluidic device including multiple channels positioned to correspond to the regions of interest of the cellular material that are opposite to the regions of interest corresponding to the first chambered microfluidic device. In a different embodiment, the channels include valves that control flow of gases or liquids through the channels of the first and second chambered microfluidic devices. In a different embodiment, the first and second chambered microfluidic device includes a dense pore. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method for selectively isolating cellular material, the method including positioning cellular material on a support, placing a photomask including a blocking region covering at least part of a blocking support over the cellular material such that the blocking region corresponds to a region of interest of the cellular material, and exposing the cellular material to photons wherein the photons penetrate the blocking support without penetrating the blocking region so that the cellular material in the region of interest is preserved and the cellular material that is not in the region of interest is damaged. In a different embodiment, the blocking support not covered by the blocking region is transparent. In a different embodiment, the method including positioning cellular material on a support, depositing a photosensitive material on the cellular material, applying a photomask including a blocking region onto the photosensitive material, exposing the photosensitive material through a light accessible region of the photomask to photons in order to generate a lithographic pattern on the photosensitive material, removing the photomask, and applying a developer to the photosensitive material in order to define an access well corresponding to a region of interest of the cellular material. In a different embodiment, the blocking region corresponds to a region of interest of the cellular material. In a different embodiment, the blocking region corresponds to a region that is not of interest of the cellular material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method for selectively isolating cellular material, the method including positioning cellular material on a support, depositing a photosensitive material on the cellular material, exposing the photosensitive material to photons in order to generate a lithographic pattern on the photosensitive material, removing the photomask, and applying a developer to the photosensitive material in order to define an access well corresponding to a region of interest of the cellular material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method for selectively isolating cellular material, the method including positioning cellular material on a support, and exposing an unwanted region of the cellular material to photons to selectively damage DNA, RNA, a protein and/or other biological component in the unwanted region of the cellular material while not exposing a wanted region of the cellular material to minimize damage to the DNA RNA, a protein and/or other biological component in the wanted region. In a different embodiment, the photons are generated by arrays of micro- and nano-lasers light-emitting diodes, or photonic crystal devices, and/or reflected onto the sample by micro-mirror arrays. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method to analyze a biological sample, the method including forming microfluidic access wells in a substrate, filling the microfluidic access wells with a reaction mixture including digestion agents and components necessary for a desired reaction, evaporating the mixture to uniformly decrease the reaction mixture level, aligning a support including cellular material facing downward on top of the microfluidic access wells such that the cellular material is exposed to the reaction mixture, vertically turning over the access wells including the support and cellular material, allowing the reaction mixture to flow by gravity to cover the cellular material, allowing the digestion agents to break down the cellular material, releasing contents from the cellular material into the microfluidic access wells, and performing the reaction simultaneously but separately in each of the microfluidic access wells. In a different embodiment, the method of claim 27, wherein the reaction includes one or more reagents for PCR, real-time PCR, RT-PCR, flow cytometry, fluorescent labeling, FRET, DNA sequencing, protein-protein interaction assays, immunoassays, protein-nucleic acid assays. In a different embodiment, the signal detection is accomplished by scanning a completed reaction using a fluorescence scanner and/or a fluorescence microscope. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method to analyze a biological sample, the method including positioning cellular material on a first support, applying a maskless microisolation apparatus including one or more randomly placed first access wells, filling the one or more first access wells with a reaction mixture including digestion agents and components necessary for a desired reaction, allowing the digestion agents to digest the cellular material thereby releasing cellular contents into the reaction mixture, inactivating the digestion agents, filling one or more second access wells in a second support corresponding to the one or more first access wells with an analytical reaction mixture, evaporating a fraction of the mixture to uniformly decrease the reaction mixture level, aligning the first support on top of the second support such that the one or more first access wells face the one or more second access wells, securing the first and second support, inverting the secured support to allow the analytical reaction mixture to contact the cellular contents, and performing a reaction simultaneously but separately in each well of the array. In a different embodiment, the reaction the reaction includes one or more reagents for PCR, real-time PCR, RT-PCR, flow cytometry, fluorescent labeling, FRET, DNA sequencing, protein-protein interaction assays, immunoassays, protein-nucleic acid assays. In a different embodiment, the signal detection is accomplished by scanning a completed reaction using a fluorescence scanner and/or a fluorescence microscope. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method to isolate a region of interest of cellular material, the method including positioning cellular material on a support, depositing a negative photosensitive material on the cellular material, capturing an image of the cellular material through the negative photosensitive material as reflected on a processing mirror or minor array, positioning the processing mirror or programming the minor array such that photons are directed to the negative photosensitive material over an unwanted region of the cellular material to laminate the negative photosensitive material over the unwanted region of the cellular material while leaving a region of interest of the cellular material non-laminated, and removing the negative photosensitive material that has not been laminated so that the region of interest of the cellular material is exposed while the unwanted region of the cellular material is sealed. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method to isolate a region of interest of cellular material, the method including positioning cellular material on a support, capturing an image of the cellular material as reflected on a processing minor or mirror array, positioning the processing mirror, or programming a mirror array, such that photons are directed to an unwanted region of the cellular material to damage DNA in the unwanted region of the cellular material while leaving DNA in a cellular region of interest intact. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method for making a micro-isolation apparatus, the method including positioning cellular material on a support, identifying an unwanted region of interest of the cellular material, converting a selection into a digital image, transferring the digital image to a plate including a layer of photosensitive material over a metal, using a laser to trace a digital mask on the photosensitive material, developing the photosensitive material to remove exposed photosensitive material, chemically etching the metal in the area where the photosensitive material has been removed, and removing remaining photosensitive material to produce a plate including a layer of metal in a pattern where the metal is absent corresponding to the unwanted region of interest of the cellular material and the metal remaining corresponds to a region of interest of the cellular material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method of making an active masking array, the method including positioning cellular material on a support, identifying a region of interest of the cellular material, polarizing an illumination light along a first axis, directing the illumination light through polarizer elements that are aligned or programmed in such a way that the illumination light is absorbed in the polarizer elements over regions of interest of the cellular material while allowing the illumination light to damage DNA or cellular material in an unwanted region of the cellular material to preserve the DNA or cellular material in the region of interest of the cellular material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method of making an active masking array, the method including positioning cellular material on a support, identifying a region of interest of the cellular material, and generating an active array of illuminators that target unwanted regions of the cellular material while allowing the region of interest of the cellular material to be non-illuminated. In a different embodiment, the illuminators are fiber optic cables. In a different embodiment, the illuminators are photonic circuitry. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method of analyzing a biological sample, the method including positioning cellular material on a first support, depositing a negative photosensitive material on the cellular material, applying a photomask including a blocking region onto the negative photosensitive material, exposing the negative photosensitive material to photons in order to generate a lithographic pattern on the negative photosensitive material, removing the photomask, applying a developer to the negative photosensitive material in order to define one or more first access wells, filling the one or more access wells with a reaction mixture including digestion agents and components necessary for a desired reaction, allowing the digestion agents to digest the cellular material thereby releasing the cellular contents into the reaction mixture, inactivating the digestion agents, filling one or more second access wells in a second support corresponding to the one or more first access wells with an analytical reaction mixture, evaporating a fraction of the mixture to uniformly decrease the reaction mixture level, aligning the first support on top of the second support such that the one or more first access wells face the one or more second access wells securing the first and second support, inverting the secured support to allow the analytical reaction mixture to contact the cellular contents, and performing a reaction simultaneously but separately in each well of the array. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a micro-isolation apparatus including a support, cellular material mounted upon the support, a photosensitive material deposited on the cellular material, wherein the photosensitive material includes an access well positioned to correspond to a region of interest of the cellular material, and a microfluidic device that provides access to the access well and is positioned adjacent to the photosensitive material. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, also described herein is a method for selectively isolating cellular material, the method including positioning cellular material on a support, depositing a photosensitive material on the cellular material, applying a photomask including a blocking region onto the photosensitive material, exposing the photosensitive material through a light accessible region of the photomask to photons in order to generate a lithographic pattern on the photosensitive material, removing the photomask, applying a developer to the photosensitive material in order to define an access well corresponding to a region of interest of the cellular material, positioning a microfluidic device that provides access to the access well and is positioned adjacent to a photosensitive material located above the cellular material, and analyzing cellular material from the regions of interest. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a method for selectively isolating cellular material, the method including positioning cellular material on a support, depositing a photosensitive material on the cellular material, exposing the photosensitive material to photons in order to generate a lithographic pattern on the photosensitive material, removing the photomask, applying a developer to the photosensitive material in order to define an access well corresponding to a region of interest of the cellular material, positioning a microfluidic device that provides access to the access well and is positioned adjacent to a photosensitive material located above the cellular material, and analyzing cellular material from the region of interest. In various embodiments, the cellular material is solidified liquid tissue. In various embodiments, the liquid tissue is blood, cerebrospinal fluid, bone marrow, lymph fluid, interstitial fluid, plasma, or similar liquid tissues known to one of ordinary skill.

In another aspect, further described herein is a solidified liquid tissue is completely encapsulated inside a microfluidic device, to allow for full surface access. Solidified liquid tissue encapsulation captures a solidified liquid tissue of interest between two separate microfluidic devices, which allow simultaneous access to two surfaces. If the slice is sufficiently thin, fluidic communication is ensured through the slice. Such communication allows more efficient and reliable extraction of desired samples, as a resuspension liquid can be used to push desired material out of the solidified liquid tissue matrix. This approach allows extraction of desired material by microfluidic/hydraulic means without the need for more aggressive chemical treatments. In an embodiment, solidified liquid tissue encapsulation allows the use of 3D polymerization for in-situ chip construction around the 3D solidified liquid tissue sample. This can be done, e.g. by using direct laser writing and 3D rastering to build the desired architectures such that the monomer material for the chip can be spread thick over the solidified liquid tissue sample. The laser can then polymerize the chip material in the desired shape over the solidified liquid tissue sample. The solidified liquid tissue is completely submerged in a monomer, while a 3D chip is built around it, thus allowing microfluidic access to the sample from all directions. In some embodiments, performed according to this approach, the photocurable polymer of the chip material itself can be used as a photosensitive material, wherein a solidified liquid tissue slice can be placed inside the polymer prior to 3-D photopatterning (e.g. before a 3-D photopatterning commences). In an embodiment, multiple areas of interest are addressed with individual channels without masking. Individually and collectively controlled arrays of microvalves, which allow the same architecture to address a customizable subset of chambers of access loci within the matrix, can provide the ability to match particular regions of interest on the particular solidified liquid tissue sample. A chip with a dense pore matrix allows the differential opening of particular pores for a short time such that only material confluent with the pore would flow from the sample into the chip for analysis without masking.

In another aspect, further described herein is an embodiment provides solidified liquid tissue micro-isolation by microfluidic matrices for parallel analysis of subsamples with preserved morphological context. According to an embodiment, microfluidic matrices with highly parallel single-cell analysis is based on the combination of a nanofabricated microfluidic matrix and a solidified liquid tissue section. This allows a matrix of millions of microfluidic wells to be filled with biochemical reagents and contacted to a solidified liquid tissue section deposited on a support. For example, each well can contain Proteinase K and sequencing reagents. The Proteinase K digests the solidified liquid tissue and releases the contents of each cell into its adjoining well, where they mix with the PCR reagents by diffusion. The entire system is contacted to a thermally controlled aluminum plate, to perform standard or isothermal PCR. Mutant genes can be amplified by appropriate selection of primers and reported by fluorescent probes. Signal detection is done by scanning the entire slide on a fluorescence scanner or by fluorescence microscopy performed one sector at a time followed by digital assembly. The result is a highly parallelized single-cell (genetic) analysis of the entire solidified liquid tissue.

In a different embodiment, releasing the cellular content of the solidified liquid tissue can be performed by digestion of the solidified liquid tissue performed using Proteinase K or other techniques of chemical digestion that allow multiple analysis of different molecules in the cellular component. For example, in an embodiment techniques can be used that allow immunoassay analysis of the extracts as well as DNA and protein analysis at various scales as it will be understandable by a skilled person.

In several embodiments, the nanomatrix technique herein described can be modified in view of the specific reagents, biological component, desired result and experimental design as will be understood by a skilled person. For example, in an embodiment, a matrix of microfluidic wells can provide access to individual cell nuclei where a two-step process allows separate biochemical reactions to occur

In one embodiment, when the surface density and size of the wells are correctly chosen, most wells will adjoin one and only one cell. In some embodiments, wherein the biological component is formed by cells, an expected optimal well size is about the same as the size of a mammalian cell (about 20 μm). However, in those embodiments, one skilled in the art would recognize that the size and spacing of the wells can be optimized to ensure that the overwhelming majority of wells contain just one cell. This would maximize purity of the sample in each well, and thus maximize specificity and reduce noise in an analytical determination.

In various photolithographic features described, it is understood that a long-pass filter indicates a device that operates to allow all light coming from the UV light having a wavelength above a certain value, e.g. about 350 nm. Long-pass filters are standard optical elements known to people skilled in the art. A long-pass filter ensures that virtually all light coming from the illumination source has a wavelength above the cut-off value of about 350 nm. The usual structure of long pass filters is a Bragg stack of layers of dielectric materials with carefully controlled thicknesses. The thickness and refractive index of each layer sets up destructive interference for a narrow band of wavelengths that are meant to be stopped. Making a stack of such layers ensures that a wider cumulative range of wavelengths is stopped by the filter. In this particular case, the cut-off value is 350 nm, because wavelengths above it are too long to damage DNA when DNA is chosen as microscale component of interest, but short enough to expose the photoresist correctly

In a different embodiment, the digital image can also be 3-D, e.g. in embodiments, when a device is provided for solidified liquid tissue encapsulation by 3-D rastering of the photocuring illumination, as described herein. A “digital mask” refers to the masking of a region of interest of cellular material based on a digital image as opposed to a physical mask, e.g. a chrome mask.

In an embodiment, solidified liquid tissues can be micro-isolated without the need for a physical mask for UV shielding. Instead, a UV laser, e.g. Heidelberg DWL66™, can be focused directly onto the necessary spots in the photoresist on top of the solidified liquid tissue for lamination or in the solidified liquid tissue itself for destruction of biological material in the solidified liquid tissue such as DNA. The resolution can be 2 microns or better, and the desired cells can be skipped in the rastering process. Different laser heads can be used for the different regions of the slide. For example, appropriate software can guide the laser with a 2-micron head around the immediate vicinity of the cells of interest, while the rest of the slide area is exposed by broader strokes, e.g. with a 30-micron head.

In another aspect, further described herein is active masking arrays utilize LCDs (liquid crystal displays). An illumination light would be polarized along one axis, while the LCD elements would be polarized along one axis to disallow and another axis to allow the passage of the UV light. The cells of interest are protected by having the corresponding elements in the array be perpendicularly aligned, while the unwanted cells would have their elements aligned in parallel with incident UV illumination.

In a different embodiment, the dynamic masking using fiber optics can be produced by arrays of LEDs (light emitting diodes). This approach allows the utilization of increasing smaller wavelengths as current technology builds LEDs at smaller wavelengths. Individually addressable elements can be built at the microscale, producing macro-sized arrays of thousands or millions of individually addressable LED elements. Such individually addressable LED elements allow respective areas on the photosensitive material to be individually photopolymerized to provide the solidified liquid tissue lamination methods described herein.

In a different embodiment, the fiber optics are used in a way similar to intensified CCD cameras. Bundles of fiber optic cables are arranged to produce an active array of illuminators. This bundle can be coupled to an LCD array at the input of illumination light, while the output is coupled to the solidified liquid tissue slide. Then the output size of each fiber can be made smaller than the input size, producing both light transduction and size reduction. In an embodiment, active masking array uses photonic circuitry to define dynamic optical arrays. A photonic circuit can in principle be built to generate an array of individually addressable optical outputs. When positioned over a solidified liquid tissue slide, the individual addressability of optical outputs provides the capability for individual UV exposure of solidified liquid tissue areas that are chosen to be discarded. In a different embodiment, the active masking array uses photonic circuitry to define dynamic optical arrays. A photonic circuit can in principle be built to generate an array of individually addressable optical outputs. When positioned over a solidified liquid tissue slide, the individual addressability of optical outputs provides the capability for individual UV exposure of solidified liquid tissue areas that are chosen to be discarded. In an embodiment, active masking array uses micro- and nano-lasers for dynamic arrays. These lasers can be fabricated in arrays, where each laser is still individually addressable. Software and electrical outputs control which laser is active, e.g. by electrical pumping or electrical control of polarization shielding against pumping illumination. Microfluidic devices can further follow a combination of morphological and functional customization. For example, in the particular technique of multi-layer elastomer microfluidics, the elastomeric layer that contacts the sample can have a photolithographically defined morphology that matches the regions of interest in the solidified liquid tissue sample, while other layers can follow a matrix or array structure built for functional programmability. Where a uniform matrix of channels overlays the wells of the regions of interest, this ensures extraction. Thus the extraction matrix can be standardized and thus produced inexpensively, while the laminating layer is kept specific to the particular solidified liquid tissue sample. One skilled in the art would appreciate that such a combination is clearly not limited to extraction alone, because a device of any processing or analytical function can be integrated with a sample-specific micro-isolation stage. In some embodiments, the specific functionality or purpose of the device can be combined with the high specificity and sample-specific customization offered by the described micro-isolation techniques. In some of those embodiments, this approach provides a low cost of standardization with a high specificity of sample-specific extraction

In some embodiments, the methods and devices described herein overcome various problems e.g. by providing a general microfluidic bottoms-up sample-specific customization method. Such a method naturally leads to rapid, parallelized, and highly specific micro-isolation of the desired cell subpopulation (e.g. cancer cells from a tumor) directly from solidified liquid tissue samples. In particular, the massively parallel technique achieves digital noise reduction in liquid tissues for detection of rare cells without bias caused by sample manipulation or preparation. A particular example includes the successful application of in situ PCR. Further, there is no signal loss, which inevitably causes poor sensitivity for detection of rare cells. A further advantage is the application of multiplexing biomarker detection to eliminate detection techniques relying upon the appearance of sometimes transient biomarkers, such as epithelial-mesenchymal transition (e.g., EpCAM).

In several embodiments, methods herein described allow convenient application in a number of methods to detect rare cancer cells. Methods herein described are not necessarily dependent on use of fresh solidified liquid tissues, and are applicable to most human cancer specimens, which may include those usually fixed in formalin and paraffin-embedded. In some embodiments, devices methods and systems herein described allow to process wanted cells (e.g. cancer cells) minimizing the background noise of unwanted cells (e.g. non-cancerous or bulk tumor cells). In some of those embodiments, devices, methods and systems herein described allow a less expensive and less labor-intensive of certain methods of the art where a trained operator must manually identify and then individually address each cell to be analyzed using an expensive and complex laser microscopy system.

Although any methods and materials similar or equivalent to those described herein can be used in the practice for testing of the products, methods and system of the present disclosure, exemplary appropriate materials and methods are described herein as examples.

### Example 1

### General Methods for Massively Parallel Detection of Rare Cells in Solidified Liquid Tissue

Briefly, we propose to divide a blood sample into thousands to millions of subsamples using microfluidic compartmentalization, and then run PCR (polymerase chain reaction) with each subsample simultaneously but separately, to identify which subsample contains a DTC or a CTC. We call this technique HemoMosaic, as it assembles a mosaic-like picture of blood samples.

In one embodiment, the blood sample is mixed with substance like agarose, which would turn into a gel at lower temperatures, or with a substance that would polymerize over time or by photoinduction. The resulting block of “blood tissue” can then be sliced into layers, each of which can be subsequently processed separately. The layer is deposited onto a flat substrate, and then a matrix of microwells is defined on top of the layer lithographically. In another embodiment, the blood can be directly distributed into a pre-made matrix of microwells, e.g. defined in a solid substrate. In all cases, a PCR mix is then distributed across the matrix, the matrix is sealed from above, and the construct is processed for PCR, e.g. in a standard flat-top PCR machine or a suitably designed machine with the same basic function. After the completion of PCR, the results are read out across the construct.

### Example 2

### Variety of Detection Techniques

The detection of the signal is tied to the type of PCR probes used. For example, it can be a hybridization/digestion probe like the Taqman technique, where primer extension results in digestion of a hybridization probe, which releases a fluorophore from the proximity of a quencher. Other ways to report the result are chemiluminescent probes, intercalation fluorescence probes, radioactive probes etc.

The premise of the technique is that DTCs and CTCs are a very small fraction of the overall number of cells in the sample—typically ˜20 CTCs/ml, while white blood cells (WBCs) are ˜100 k/ml and red blood cells (RBCs) are ˜1 B/ml. Thus any physical selection/purification technique runs a significant risk of allowing some CTCs to escape capture and subsequently detection. By contrast, our technique does not select nor does it purify, so it is reasonable to expect that it would be far less lossy as a method of CTC detection. Instead it relies on massively parallelized compartmentalization and PCR, i.e. a brute-force way of analyze the entire sample.

### Example 3

### Digital Noise Reduction Through Massive Parallelism

While the RBCs are the most numerous cells by far in the sample and comprise about half of its volume, they do not possess a nucleus and thus genomic DNA, which means in each microwell compartment of the matrix, they cannot produce noise for the PCRbased identification of the CTCs potentially present in the same well. By contrast, WBCs do have a nucleus and thus can contribute noise, but compartmentalizing the sample into millions of wells means that on average less than one WBC would be present in each microwell, most likely one or none. Thus PCR should have no problem identifying the CTC in the well, if it is present.

This method of detection is inherently digital in the sense that once the PCR is completed, any well that “lights up” would indicate just one CTC on average, so counting the number of lit wells would reveal the number of CTC's in the sample's volume. While it is possible that more than one CTC is present in the same well, the distribution of the sample across the millions of wells makes such an occurrence extremely improbable.

### Example 4

### Applications for Detection of Rare Cells in Blood

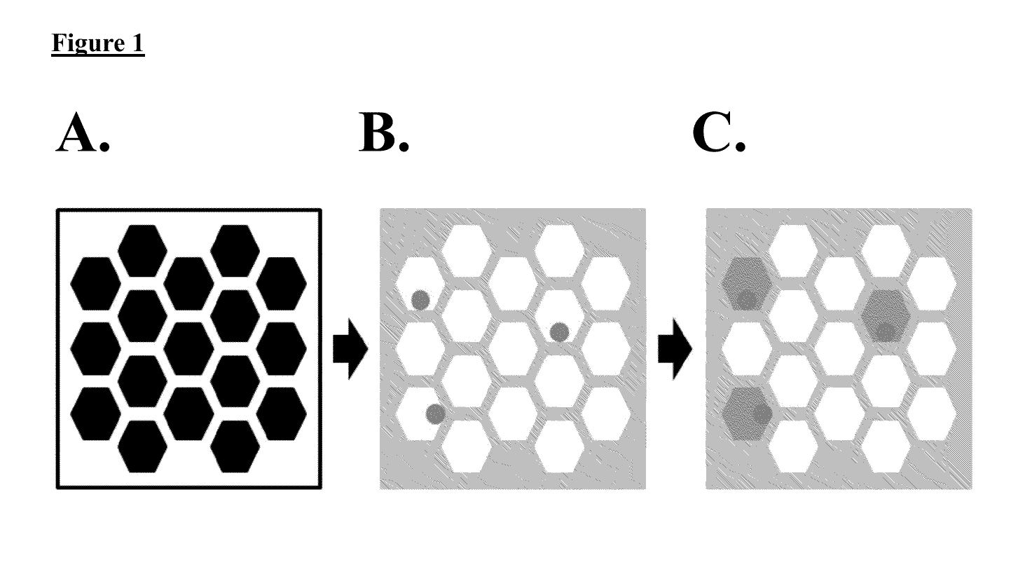

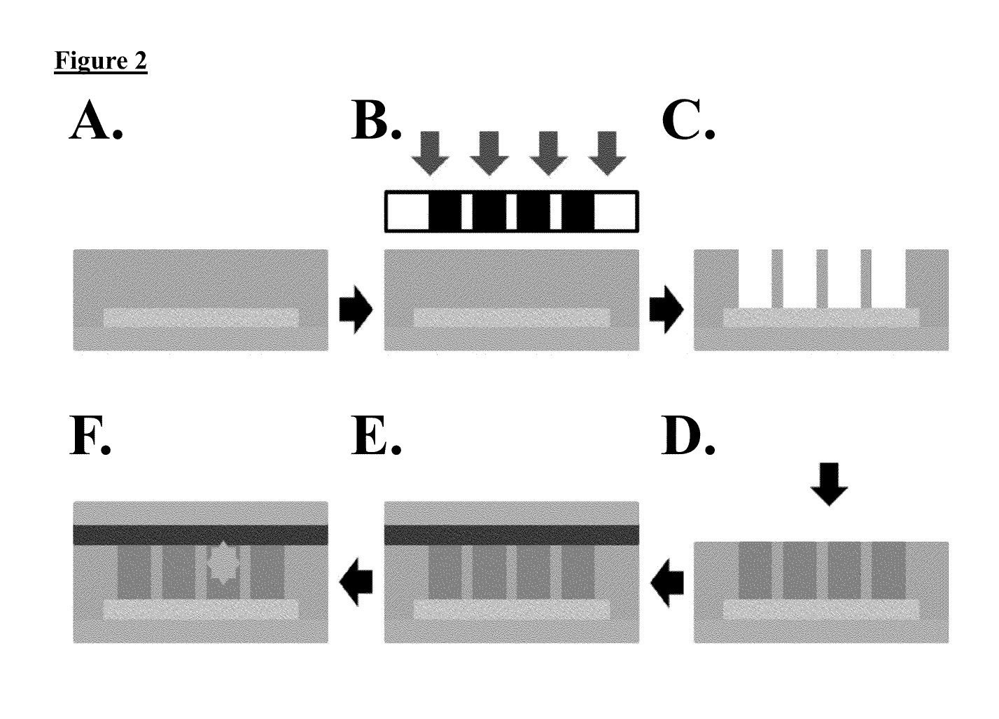

Here is a more detailed description of the workings of the system in the embodiment involving the production of a block of “blood tissue”, e.g. by mixing the blood sample with agarose to produce a gel or with a cross-linking substance to produce a polymerized material. The basic procedure of the technique is shown in FIG. 1 (top view) and FIG. 2 (side view). Briefly, photoresist (a photocurable substance, gray) is deposited onto the “blood tissue” slice (pink) located on a standard glass slide (green) (FIG. 2A). The photoresist is exposed to UV light (FIG. 2B, violet) through a photolithographic mask bearing a hexagonal matrix pattern (FIG. 1A). The dark areas in the mask absorb the UV and thus protect the photoresist under them, while the light areas transmit the UV light to the underlying photoresist. The UV-exposed photoresist cures into a hard transparent mechanically strong material, while the protected uncured photoresist is removed by organic solvent during photolithographic development. The result is a matrix of hexagonal wells on top of the tissue (FIGS. 1B, 2C).

The size of the wells would determine how many cells on average will be contained in each well. Generally, it is preferable to have a very large number of smaller wells, so that the benefit of compartmentalization and decreased noise from WBCs is maximized. However, so long as there are enough wells so that less than one WBC can be expected per well, the sensitivity of the technique is maximized.

### Example 5

### Mosaic-Like Digital Detection

FIG. 1C shows a cartoon representation of the CTC distribution, where the blue circles represent the CTCs. For simplicity of presentation, the RBCs and WBCs are not explicitly shown but are assumed to be in the white hexagonal wells.

After the wells are defined (FIGS. 1C, 2C), they are filled at the same time with the same PCR reagents (FIG. 2D, light blue). Then the matrix is sealed (FIG. 2E) with another glass slide (green) coated with cured elastomer (red) to serve as a gasket layer. The assembled chip is then processed in a standard flat-top PCR machine. If the PCR assay is color-multiplexed hybridization-digestion probes, different colors would correspond to positive results for different sequences. Note that while the assay is for real-time PCR, here we run the reaction to completion and then detect the products by fluorescence measurements (FIGS. 1C, 2F). FIG. 2C shows the expected result of the presence of the CTCs, where a single fluorescence color is used for a hybridization-digestion probe. Similarly, FIG. 2F shows just one well lit up in the cartoon representation.

### Example 6

### Coverage

Since the walls have non-zero thickness, it is inevitable that some of the sample material will end up under them rather than in a microwell. It is desirable to minimize that fraction, so as to maximize the percentage of the total sample layer area covered by the diagnostic test. This percentage can be defined as the “coverage”. A geometric calculation shows that the coverage C=1/{1+T/[R*sqrt(3)]}, where T is the wall thickness and R is the radius of the hexagon. For 20 μm-wide wells separated by 10 μm walls, C=63%. If the wells are the same but the walls are 2 μm-wide, C=90%. Such dimensions are easily achievable through the use of chrome-on-glass masks printed on a modern direct writer, and as the mask is reusable and not sample-specific, no major difficulty or expense is incurred.

It should be noted that the coverage does not have to be close to 100% for the technique to be useful. It is reasonable to expect that the CTCs are uniformly distributed in terms of being within wells or under walls. So, a simple arithmetic factor can be applied to adjust for the coverage. For example, if the coverage is 75% and 21 cells/ml are detected, then a quarter of all CTCs must not be accounted for due to the coverage, so the apparent number must be adjusted by adding a third more; then the final count would be 28 cells/ml.

### Example 7

### Hardwired Masking Using Photolithographically Defined Chrome Masks

While the massively parallel technique describe above achieves digital noise reduction in liquid tissues containing a majority of cells with little or no DNA, such as blood, extension of the above method to other liquid tissues, such as lymph fluid or bone marrow, may require additional steps in order to isolation materials of interest for detection. Unlike blood, many, or a majority of cells in these liquid tissue types include cells with DNA. As a result, dilution of the liquid tissue sample may be necessary achieve detection via massively parallel digital noise reduction, as described above. Alternatively, strategic photomasking can be applied to isolate cells of specific interest, as described below. In various embodiments, a combination of these various techniques can be applied together depending on the liquid tissue of interest.

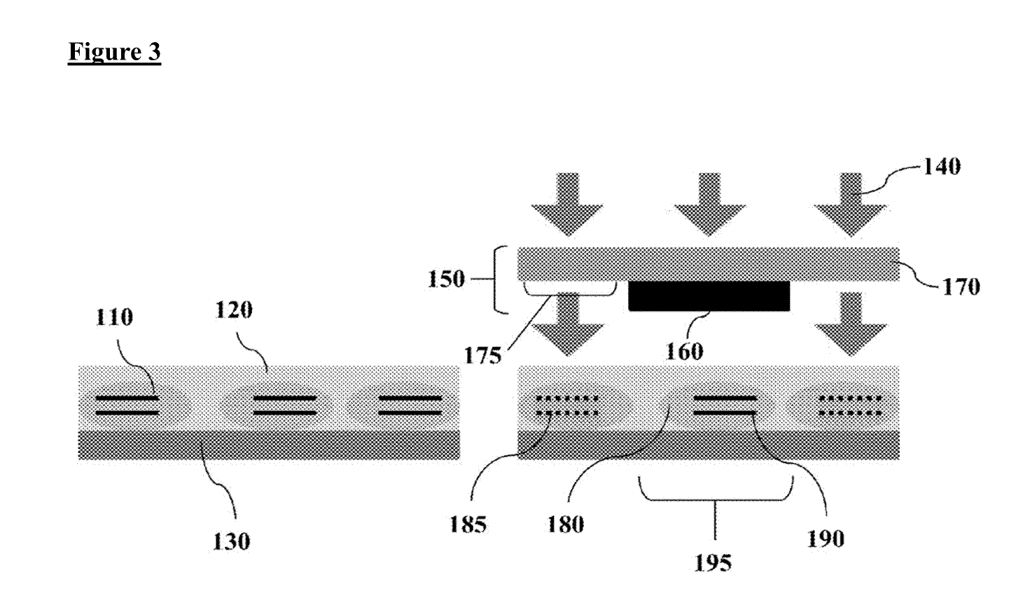

In one example, “hardwiring” of a solidified liquid tissue sample allows isolation and detection of cells in a solidified liquid tissue. Cells of interest are identified using a microscopic computerized image of the solidified liquid tissue slide and appropriate custom software, which converts the selection into a digital image. The digital mask is fed into a direct laser writer, the Heidelberg DWL66™, which transfers a digital mask onto the “positive” photosensitive material deposited on top of a chrome-covered plate, by direct writing with a resolution of 2 microns. The plate is then developed to remove the exposed photoresist, which leaves the exposed areas susceptible to chemical etching. The etching removes the unprotected chrome, and the rest of the photosensitive material is removed, e.g. by overdevelopment or exposure to a strongly alkaline solution. The remaining chrome pattern is quickly oxidized by atmospheric exposure, typically within 30 sec, which produces a chrome mask specific to the particular solidified liquid tissue sample. An exemplary hardwired masking using photolithographically defined chrome masks is illustrated in FIG. 3.

FIG. 3 illustrates exemplary hardwired masks. Cells (110) inside a solidified liquid tissue slice (120) on a solidified liquid tissue support (e.g. glass slide) (130) are exposed to ultraviolet (UV) light (140) through a photomask (150) including blocking regions (e.g. chrome regions) (160) patterned on a transparent blocking support (e.g. glass slide) (170). The blocking regions (160) are patterned in correspondence to cells of interest (180). The illuminating UV light (140) passes through a region of the transparent blocking support that is not blocked (175) and is prevented from exposing an area (195) protected by the blocking region (160). DNA in exposed cells is destroyed (185) but protected DNA inside the cells of interest is preserved (190).

### Example 8

### Combinations of Features

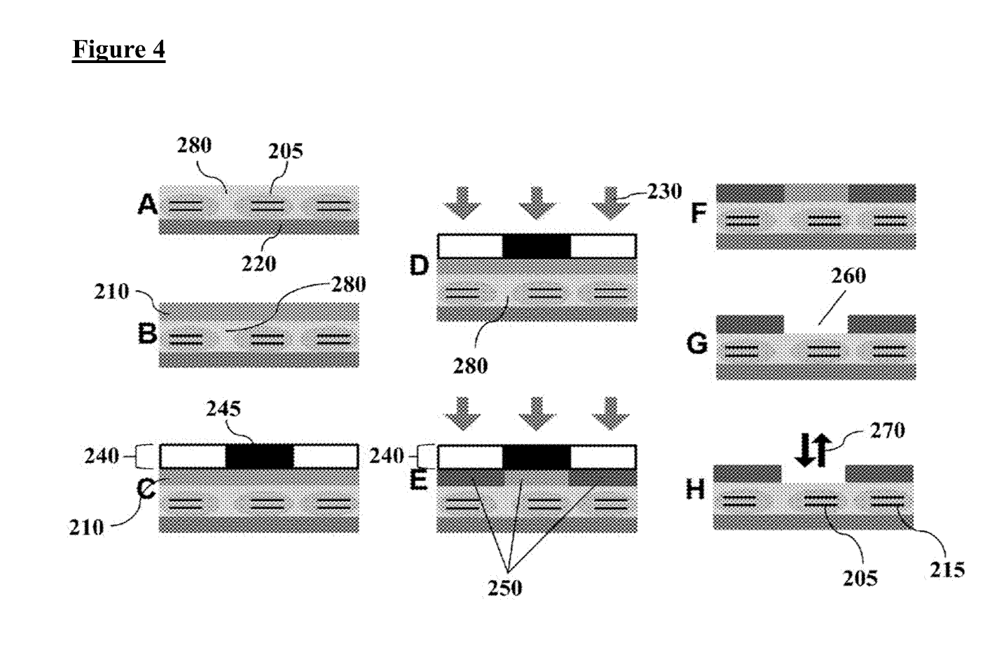

Further description of the various features in different embodiments are described in combination. In accordance with various embodiments, FIG. 4 shows a cross-sectional view of a series of steps where solidified liquid tissue isolation or masking is performed through lamination with a photosensitive material. (A) A solidified liquid tissue (280) containing a cell of interest (205) is fixed on a solidified liquid tissue support (220). (B) A photosensitive material (210) is then deposited onto the solidified liquid tissue (280). The photosensitive material can be deposited on the solidified liquid tissue, e.g. by simple application, by spinning the substance down on a spincoater, by kinetic mounting, or by using spacers (e.g. microspheres of fixed dimensions) and mechanical contact with a flat surface. (C) A photomask (240) including a blocking region (245) is then applied onto the photosensitive material (210). (D) The solidified liquid tissue (280) is then exposed to UV light (230) through the photomask (240) and the photosensitive material (210). (E) Photoexposure through the photomask (240) produces a lithographic pattern (250) inside the photosensitive material (210). (F) The photomask (240) is removed. (G) A developer is applied (not shown) to remove non-cured sections of the photosensitive material, which leaves the areas of interest (260) open to interaction with the outside world. (H). The cell of interest (205) is unprotected and subjected to removal (270) for subsequent biochemical analysis (e.g. extraction or in-situ measurements) whereas unwanted cells (215) are left inaccessible.

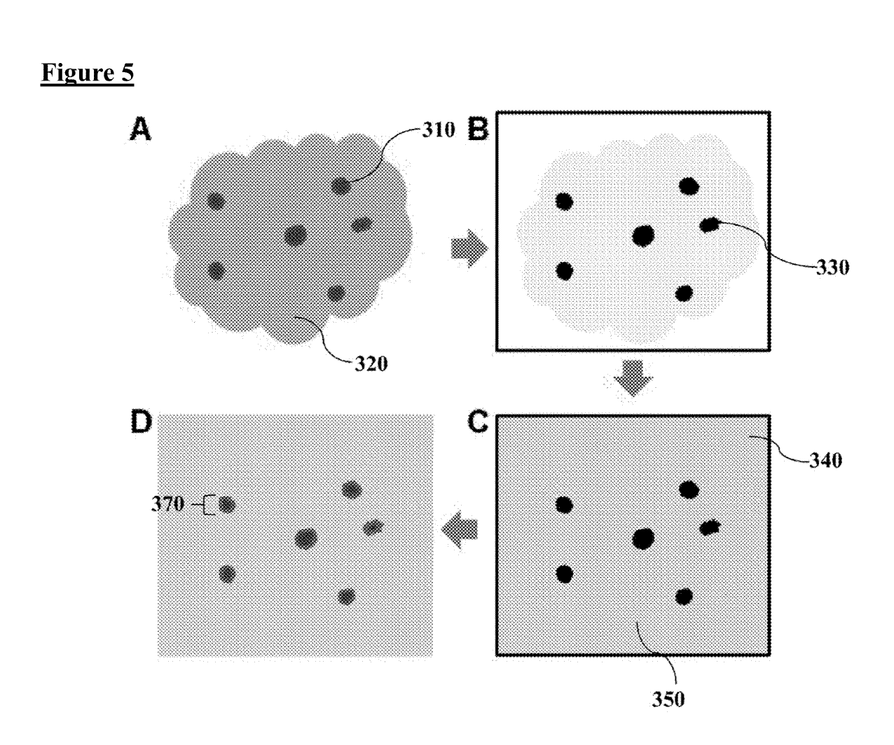

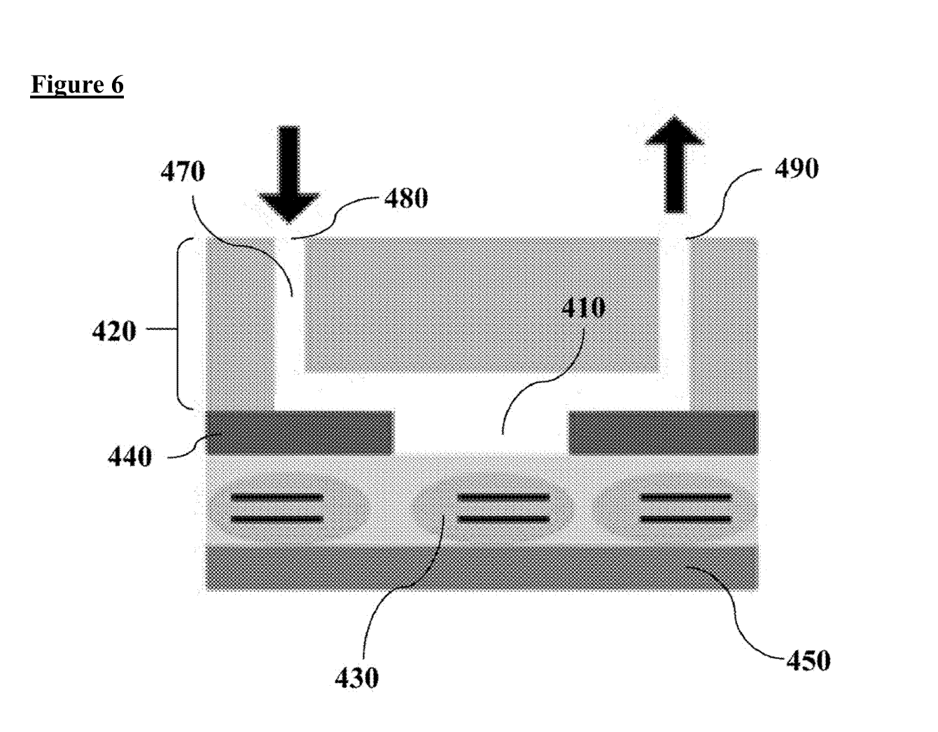

In further accordance with various embodiments, FIG. 5 shows a top view of an embodiment in which solidified liquid tissue isolation targets multiple areas of interest occurring by lamination with a photosensitive material. (A) Clusters (310) of potential cancer cells within a solidified liquid tissue sample (320) are selected. The clusters provide a plurality of cells, each of which are targeted in a manner described in FIG. 6. (B) The selection is reflected in a photomask (either hardwired or dynamically defined) of black spots (330). (C) A photosensitive material (340) is deposited onto the solidified liquid tissue sample (320) and a solidified liquid tissue support (not shown); the photomask (330) is aligned on top, and UV light (not shown) is directed through the photomask (330) to expose the photosensitive material (340) over unprotected unwanted regions. (350) (D) The solidified liquid tissue slide (not shown) is treated (e.g. with a developer), which removes unexposed photosensitive material above the areas of interest. Defined access wells (370) in the photosensitive material (340) ensure that only the wanted areas can be extracted with suitable methods (e.g. chemically) for further analysis.

In further accordance with various embodiments, FIG. 6 shows a cross sectional view where a solidified liquid tissue is integrated with microfluidic elements. access wells (410) defined in a photosensitive material (440) over cells of interest (430) placed on a solidified liquid tissue support (450) can be accessed microfluidically by producing and aligning a chambered microfluidic device (e.g. a microfluidic chip) (420) including channels (470) having an input (480) and an output (490).

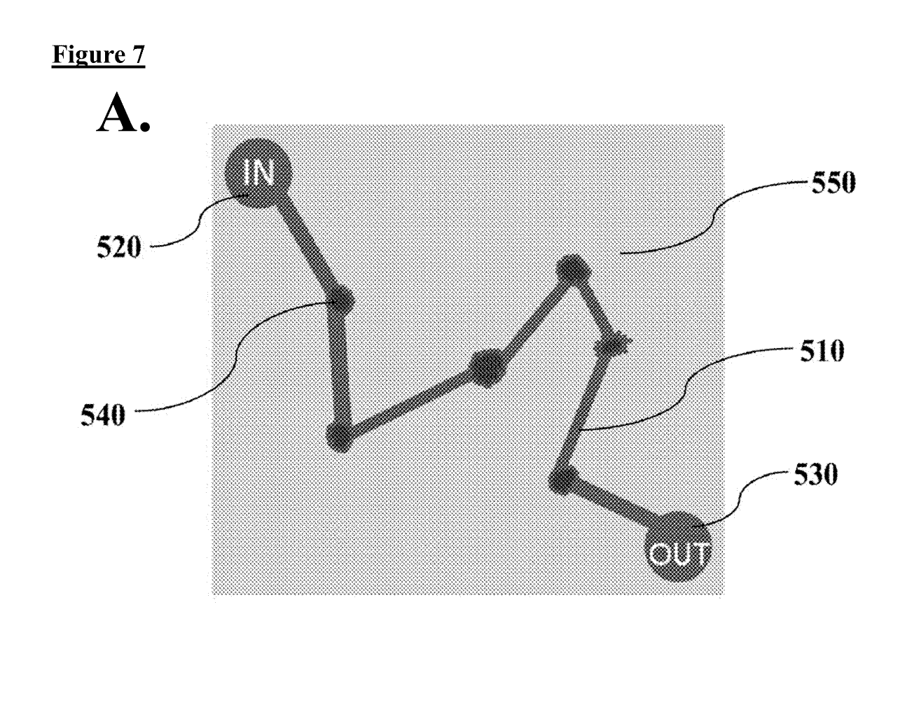

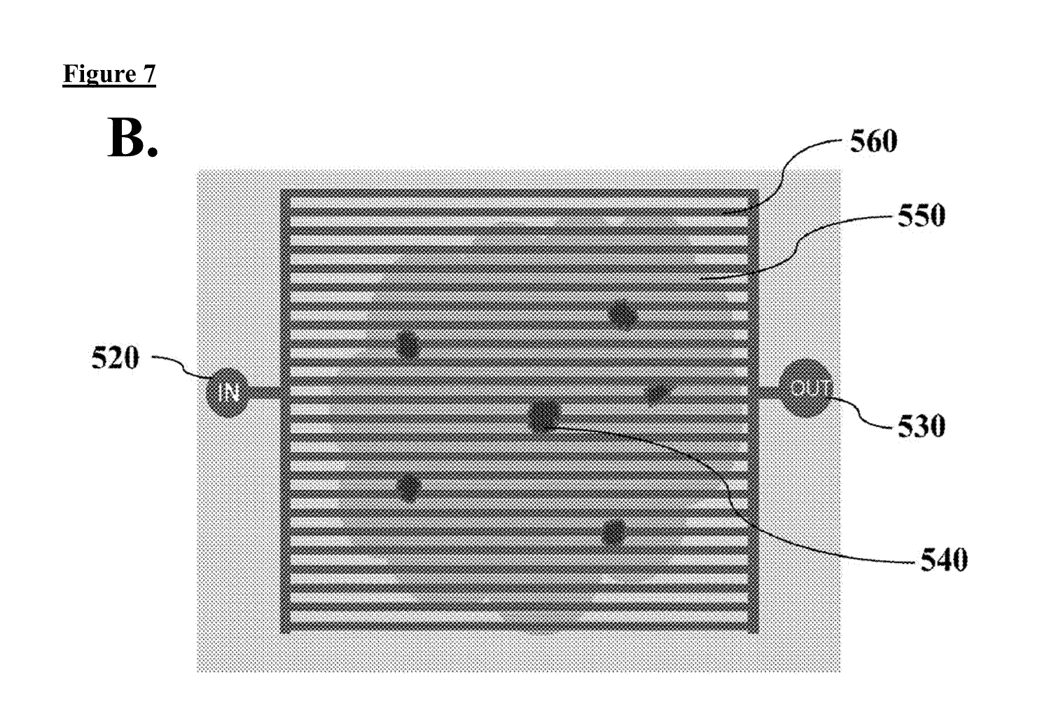

FIG. 7 (A) is related to FIG. 3, and illustrates a top view of a customized chambered microfluidic device. (B) is related to FIG. 3, and illustrates a top view of a standardized chambered microfluidic device. A laminated solidified liquid tissue sample (550) is integrated with a chambered microfluidic device (as shown in FIG. 6), whose channels (510) connect to chambers (as described in FIG. 6) over the laminated solidified liquid tissue's cellular areas of interest (540). An input (520) allows introduction of components necessary to collect extracts into a single output (530). FIG. 7A shows a single input and output, although multiple inputs and outputs are possible. In this particular approach, the entire chip is customized to the extraction needs of the particular sample, although as described herein, multiple approaches are possible.

### Example 9

### Complete Encapsulation of Cellular Material of Interest

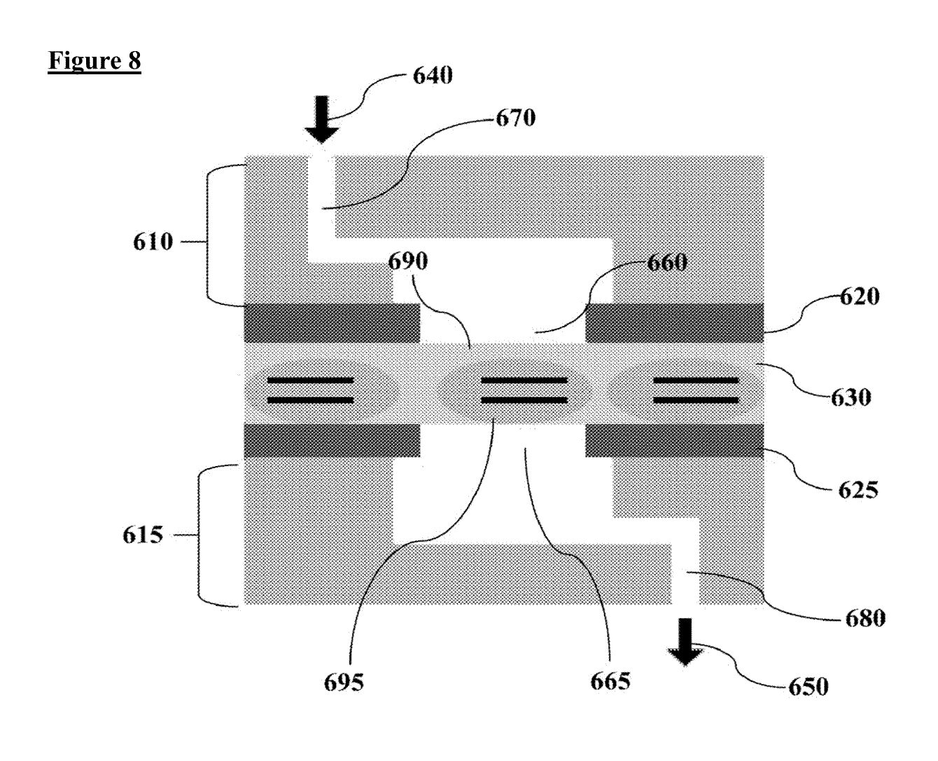

FIG. 8 illustrates an embodiment where complete encapsulation is shown. A solidified liquid tissue slice (630) covered in a photosensitive material both above (620) and below (625) the solidified liquid tissue slice and defined by access wells both above (660) and below (665) the solidified liquid tissue slice that can be integrated with microfluidic devices (e.g. microfluidic chips) positioned above (610) and below (615) the cells of interest and having chambers from both above (670) and below (680) the solidified liquid tissue slice. This architecture allows a more efficient input (640) and output (650), while an area of contact (690) can be doubled for better access to the cells of interest (695).

### Example 10

### Maskless Microfluidic Encapsulation

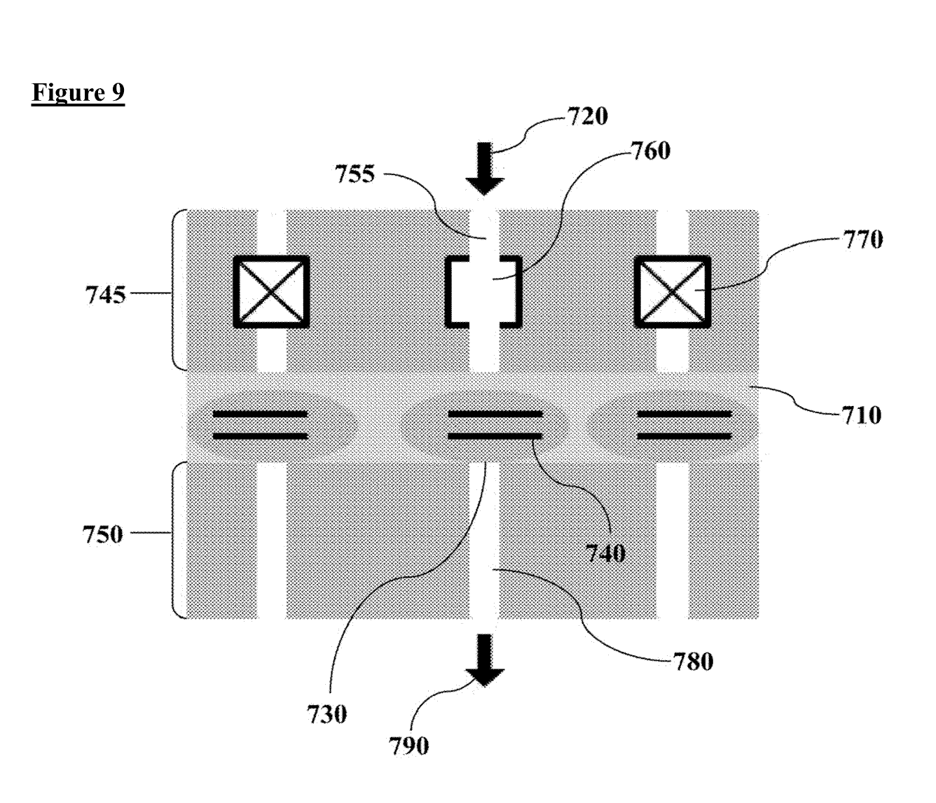

Maskless microfluidic encapsulation is shown in FIG. 9. A solidified liquid tissue slice (710) is encapsulated in a dense pore matrix microfluidic device both above (745) and below (750) the solidified liquid tissue. Individually addressable valves that are closed are shown marked with an “X” (770) as opposed to valves that are open (760) to allow flow through the open channel (755). The open valve (760) forces a pressure drop that ensures input flow (720) and forces material from a cell (740) through a pore (730) into an output channel (780) and through an output (790) for analysis.

### Example 11

### Matrix of Microfluidic Wells

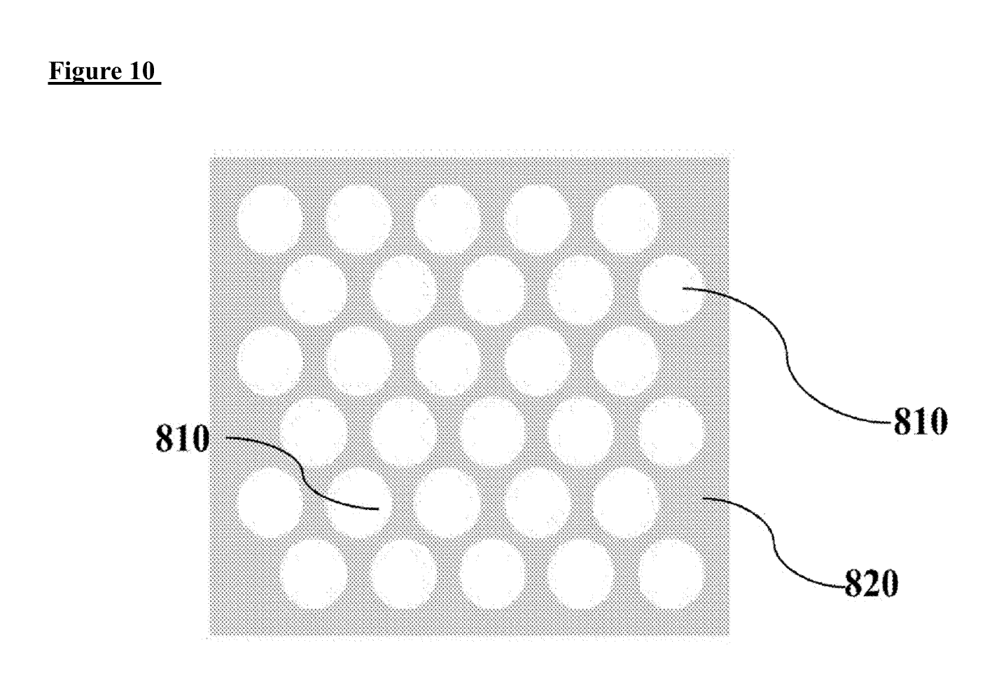

An exemplary matrix of microfluidic wells according to an embodiment herein described is illustrated in FIG. 10, which shows a top view of access wells (810) inside a matrix (820) (built in e.g. glass silicon, or silicon-on-insulator). Access wells (810) can be defined, for example, by spreading a photoreactive material on a substrate, exposing the photoreactive material to UV light through a photomask, developing the photoreactive material, and etching the exposed areas. Following removal of the photoreactive material, the result is defined access wells in the substrate with the same geometry as the photomask. The term “dense-pore matrix” as used herein refers to a matrix having dense pores.

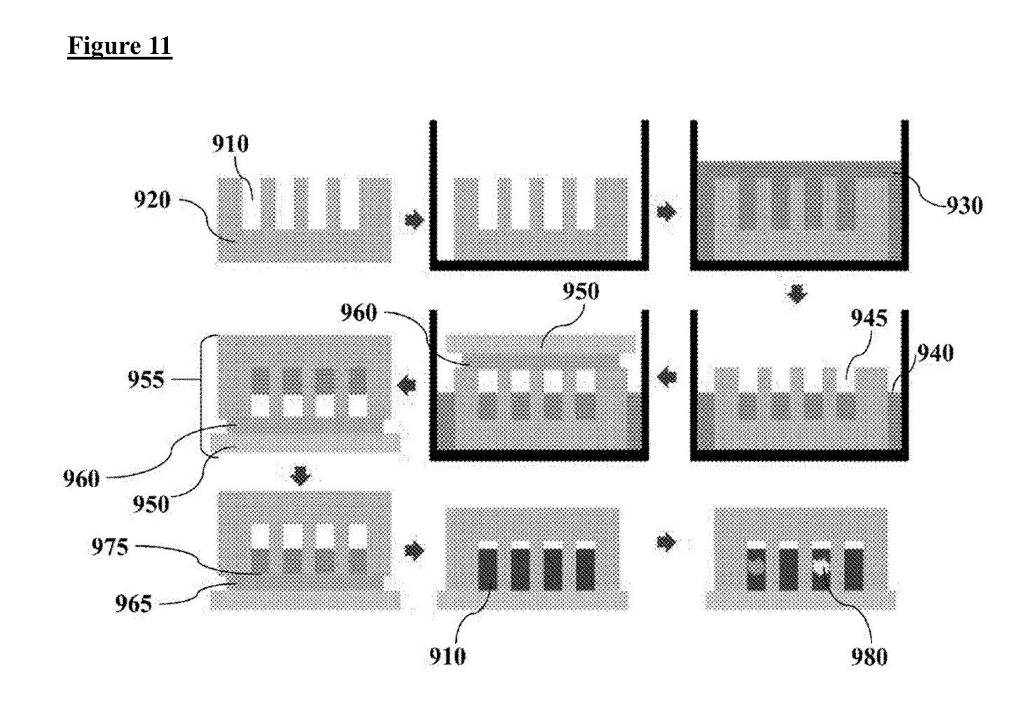

FIG. 11 shows an exemplary illustration of how a matrix of microfluidic wells can provide access to individual cell nuclei for independent reactions. Microfluidic access wells (910) are defined in a substrate (920) (e.g. glass or silicon). Next, the access wells (910) are filled with a fluid mixture (930) containing digestion and reaction agents (e.g. Polymerase Chain Reaction (PCR) reagents, and fluorescent probes). Next, evaporation is performed to uniformly decrease the fluid mixture level (940), leaving space at the top (945) of the microfluidic access wells. Next, a solidified liquid tissue support (950) is aligned on top with a solidified liquid tissue slice (960) facing downward. Next, an assembled construct (955) is clamped together and vertically turned over, allowing the fluid mixture to flow by gravity and cover (975) the solidified liquid tissue. Next, the digestion agents contained in the fluid mixture break down the solidified liquid tissue (965), releasing the cellular contents into the microfluidic access wells (910). The reaction reagents within the fluid mixture complete the reactions and fluorescent probes (980) reveal results.

### Example 12

### Digestion Using Proteinase K

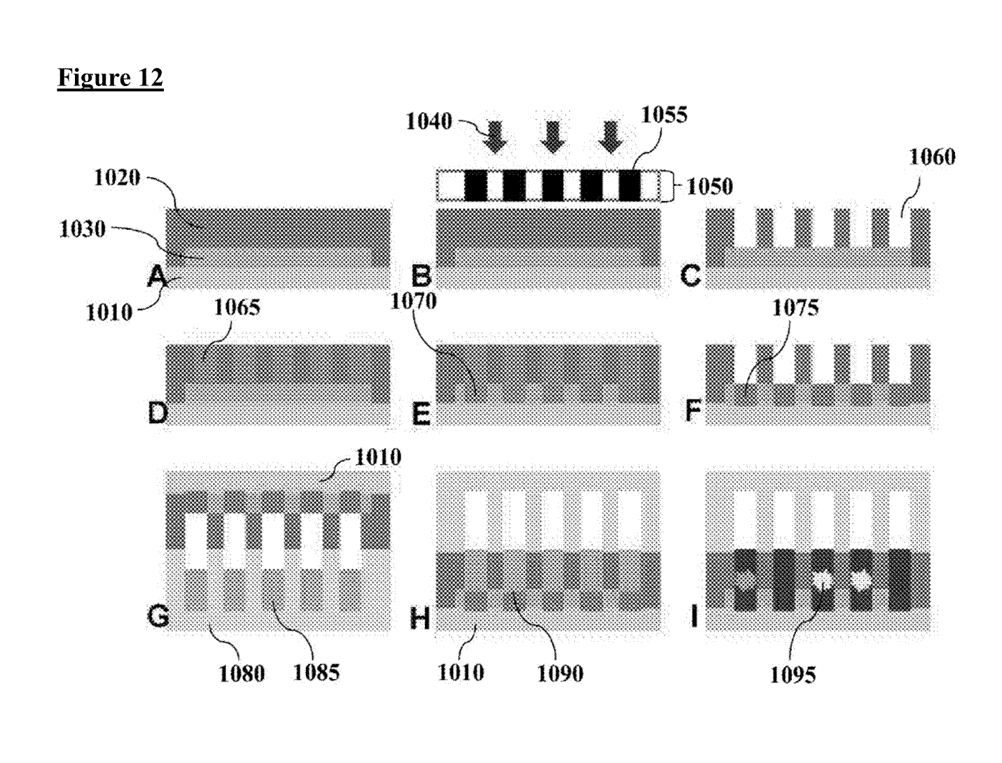

By way of example, FIG. 12 shows a process in which cells can be digested with Proteinase K prior to a PCR reaction. In the illustration of FIG. 12 solidified liquid tissue (1030) is placed atop of a support (1010) and a photosensitive material (e.g. negative photoresist) (1020) covers the solidified liquid tissue (Panel A). Next, a photomask (1050) having—blocking regions (1055) is placed over the photosensitive material and exposed to UV light (1040) (Panel B). Next, the photomask is removed and an organic solvent developer removes the photosensitive material from the unprotected areas, leaving defined access wells (1060) (Panel C). The defined access wells are filled with solution containing Proteinase K (1065) (Panel D), which digests exposed solidified liquid tissue (1070) and releases the DNA into the solution in each well (Panel E). Heating deactivates the Proteinase K and lyophilizes DNA in place in each respective well (1075) (Panel F). Next, a corresponding matrix of wells is etched in a well support (e.g. silicon, glass, or silicon-on-insulator) (1080), which is filled a solution containing PCR reagents (1085) (Panel G). The assembly is then mechanically secured together (e.g. clamped) providing water-tightness between compartments, and then turned over, allowing the PCR solution to resuspend the lyophilate within each well (1090) (Panel H). PCR can proceed simultaneously yet separately, in which fluorescent probes (1095) reveal results of the reaction (see starbursts Panel I). Data acquisition can be performed e.g. on a fluorescence scanner or by an optical fluorescence microscope, where the wells are optically accessed by the side of the glass slide in panel I.

### Example 13

### Active Arrays of Masking Material

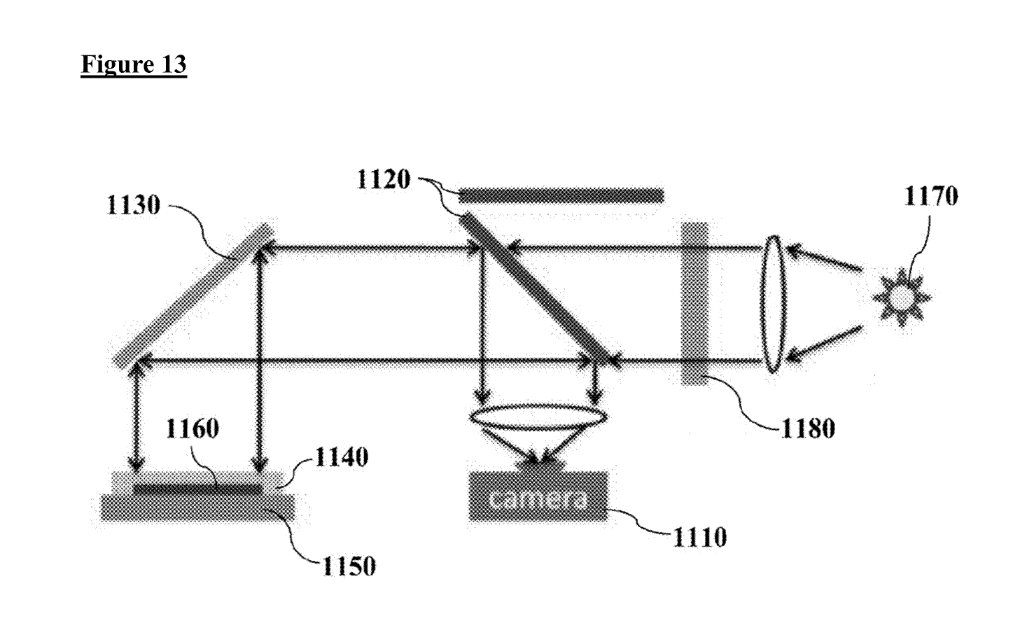

In an embodiment, an active array of masking material replaces a physical mask with micro-minor arrays. As illustrated in FIG. 13, which is a dynamic process allows the targeted positioning of solidified liquid tissue selection. First, cells and/or solidified liquid tissue (1160) are shown to an operator and a camera (1110) takes an image of cells and/or solidified liquid tissue through an adjustable mirror (1120), a Digital Light Processing (DLP) minor (1130) and a photosensitive material (1140). The image shows the cells and/or solidified liquid tissue (1160) placed on a solidified liquid tissue support (1150). Upon observation of the image, an area of interest is selected by programmable patterning the DLP mirror (1130). Second, the adjustable minor (1120) is adapted to be positioned accordingly so that UV light from a lamp (1170) can be directed through a long-pass filter (1180) and through the programmed DLP mirror (1130) onto the photosensitive material (1140) and directed to destroy the DNA of the cells in the region of interest (1160).

In the exemplary system of FIG. 13 the long pass filter is for the solidified liquid tissue lamination method, which necessitates the exposure of the photoresist which becomes the laminate. People skilled in the art (e.g. optics and engineering) understand all the possible variations of long-pass filters in devices, methods and filters herein described.

### Example 14

### Application of Lamination Approach to Adrenal Gland Tissue Slides

The lamination technique was applied to adrenal gland tissue slides prepared by routine clinical methods. Photoresist SU8-2005 was deposited onto a tissue by spinning the slide on a WS-400B-6NNP/LITE spincoater. The slide was pre-baked at 65° C. Next, the slide was exposed to UV filtered with a 368-nm high-pass filter at an MA-6 mask aligner, through a chrome-on-glass mask bearing the pattern of a USAF 1951 resolution chart. The chart was chosen as a mask to provide an easily identifiable reference in terms of size of the defined features in photoresist on top of the tissue.

The slide was then post-exposure baked at 95° C. and developed in SU8 developer, which contains organic solvents. Finally, each slide was characterized on a profilometer (Alpha-Step 500) to measure the height of the fabricated features. Tissue slice thickness was measured up to 5 μm tissue, while the photoresist layer was about 7 μm high. Dimension defined on the tissue can already be focused as narrowly as 12 μm width, which is smaller than a typical mammalian cell (20 μm).

Importantly, tissue section is essentially unchanged after photolithography, except for the discoloration of unmasked areas due to the leeching of the hematoxylin and eosin staining by the organic solvent of the photoresist developer. Some of this discoloration extends under the mask, likely because the organic solvent is a very small molecule that can penetrate through the tissue to reach the masked areas. An alternative explanation is that the dye can diffuse out into the wells during the digestion and extraction process, leaving the areas of immediate proximity to the wells. It is noted however that the nuclei remain in the unwanted areas but are extracted from the wanted areas—therefore, the unwanted DNA cannot diffuse out the way the dye can.

The laminated areas of the tissue appear far brighter than the exposed tissue because the refractive index of the photoresist matches the refractive index of the tissue better than air, while the photoresist also mechanically smoothens the surface roughness of the tissue. Thus surface light scattering and refractive divergence are significantly reduced, and the intensity of the detected light is increased over the laminated areas, in comparison to non-laminated tissue.

To extract the exposed tissue, a drop of extractions solution (10 mM Tris-HCl, 2 mM EDTA, pH 8.0, with 10 mg/ml Proteinase K) is placed on top of the masked slide and incubated at 56° C. in a humidity chamber. The Proteinase K digests the tissue, releasing the DNA into solution, which is then suitable for amplification by PCR. The slide after digestion shows the removal, with sharp boundaries defined by the mask, because Proteinase K is a large protein and thus unable to diffuse through the tissue. Digestion is less efficient with smaller features, because the photoresist is hydrophobic and so surface tension works as counter pressure against the entry of the extraction solution into the smaller holes.

### Example 15

### Preliminary Results

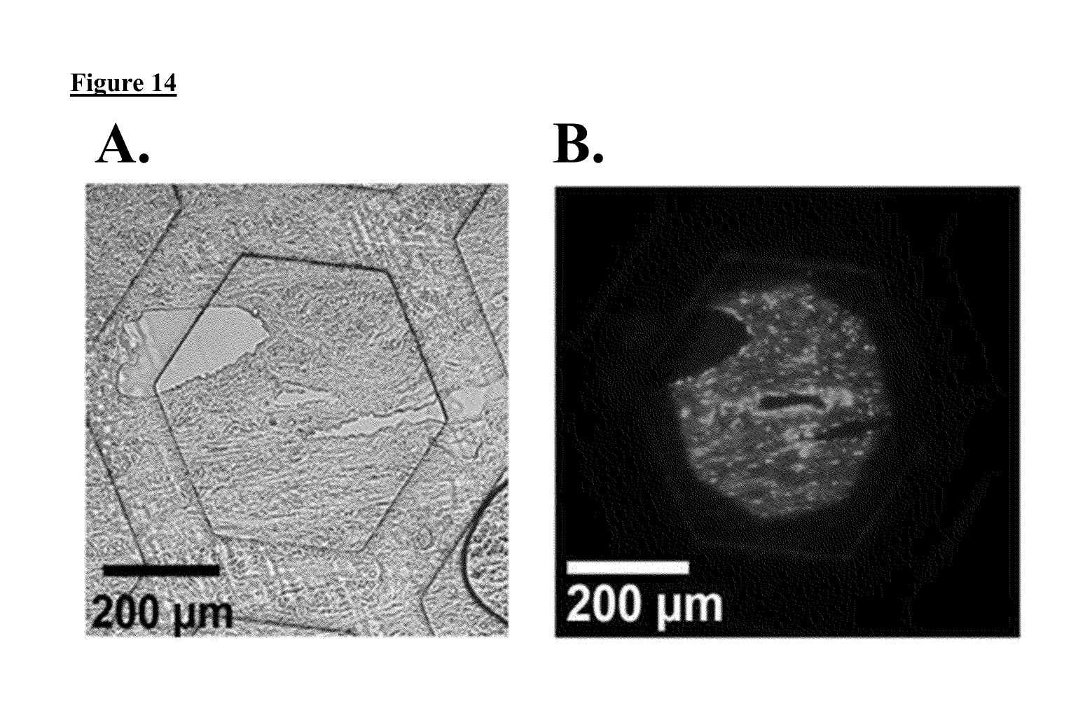

As a preliminary study system colorectal cancer tissue is used, but the teachings described herein are applicable to a polymerized and gel-formed blood sample, after incorporating the massively parallel digital noise reduction techniques described earlier. A matrix can be built on top of colorectal cancer tissue without damage to the tissue (FIG. 14A). This is so because both manipulating the tissue and developing the photoresist make use of organic solvents. We have also shown that PCR amplifies DNA within the matrix and that the amplification is detectable by fluorescence microscopy within the wells. FIG. 14B shows an example of the fluorescence image of a well with tissue.

### Example 16

### In Situ PCR Reactions

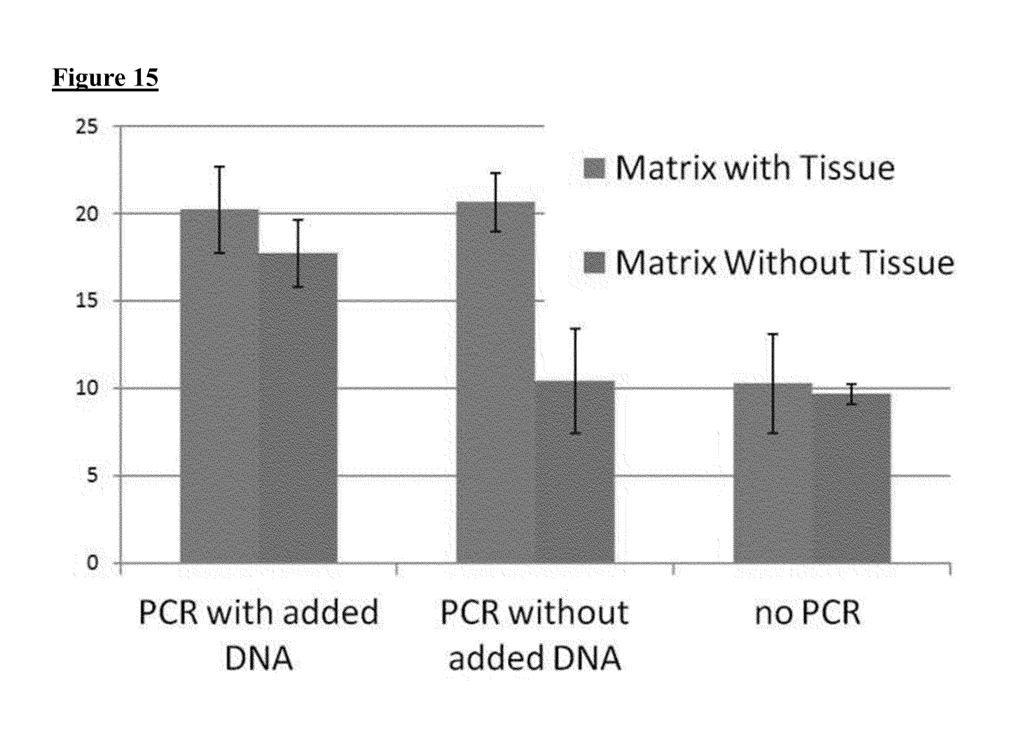

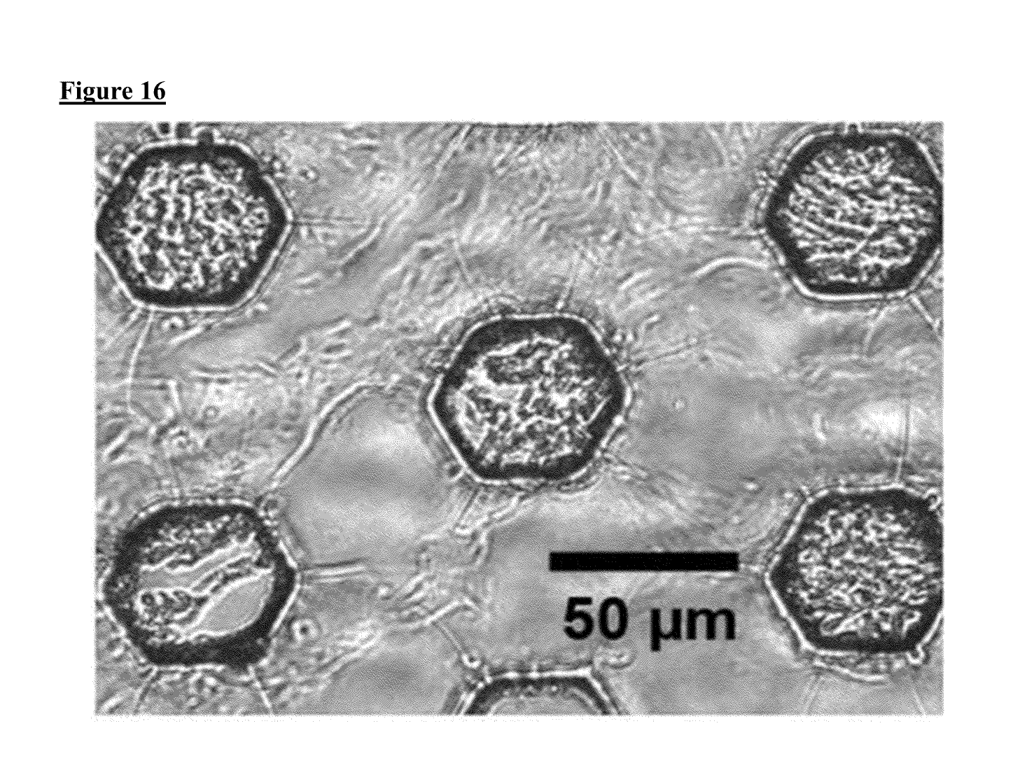

In-situ PCR amplification from the DNA of the tissue is now achieved using CRC tissue slides and a standard PCR machine. Analyzing three general cases: a) positive control: PCR reagents and DNA added to matrix and then performed PCR; b) true experiment: same as positive control but without added DNA; c) negative control: PCR reagents added but PCR not performed. In each case, there were two subcases: tissue present in the wells and tissue absent in the wells. In each of the resulting six experimental sets, about 30 fluorescence images were taken, and the mean and standard deviation were calculated. The results are shown in FIG. 15.

In the negative control case (FIG. 15, right pair of columns), the tissue and no-tissue subcases both produce about the same low signal. In the positive control case (FIG. 15, left pair of columns), the tissue and no-tissue subcases both produce about the same high signal. In the true test (FIG. 15, middle pair of columns), the tissue subcase produces about the same high signal as the positive control, while the no-tissue subcase produces about the same low signal as the negative control. Thus there is no DNA contamination (otherwise true test no-tissue would produce high signal), so the high signal in the true test tissue subcase indicates successful PCR amplification from the DNA of the tissue inside the matrix. Clearly, these results demonstate successful in-situ PCR based on the described apparatus and methods.

Our preliminary results (FIGS. 14, 15) were produced on a standard fluorescence microscope (Olympus IX70 with Hg lamp illumination) after running the PCR on the slide sandwich (FIG. 2F) inserted in a standard PCR machine. However, complete maps with up to a million wells cannot be practically assembled by taking individual well images like FIG. 14B using this setup. Instead, one can employ a confocal fluorescence microscope to scan the sample, as well as fluorescence scanners used in clinical pathology. It would be a relatively simple matter to engineer a parallel-readout optical system for high-speed high-quality fluorescence scanning of the post-PCR slides. For example, the chip of an intensified CCD can be coupled to the slide to offer objective free high-numerical-aperture imaging that would be fast, parallel, and high-throughput.

### Example 17

### Computer-Assisted Design Incorporating Electronic or Computer Board Manufacturing Techniques

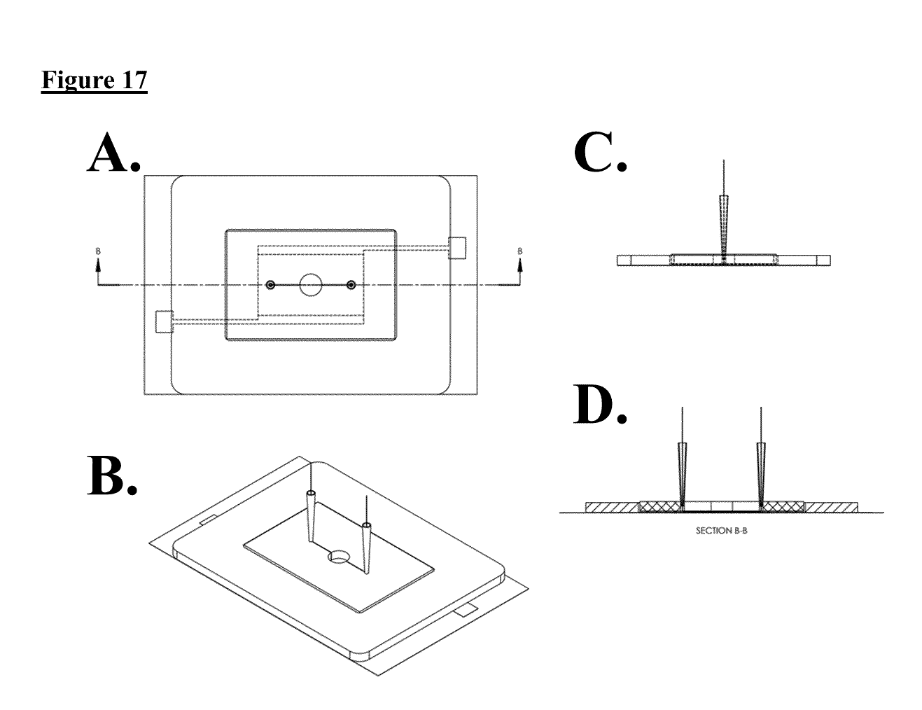

In accordance with various embodiments, the described apparatus can be modified to easily have flowable sample interact with electronics using commonly known electronic or computer printed circuit board manufacturing techniques. These standard processes utilize materials such as FR4, copper foil, Kapton and various adhesives to build three dimensional structures. An illustrative example of assembling such a device using computer-assisted design (CAD) is show in FIG. 17. Pockets may be defined by etching various layers, or by cutting a void in the material layers and then assembling, creating a pocket and channels for fluid or flowable liquid.

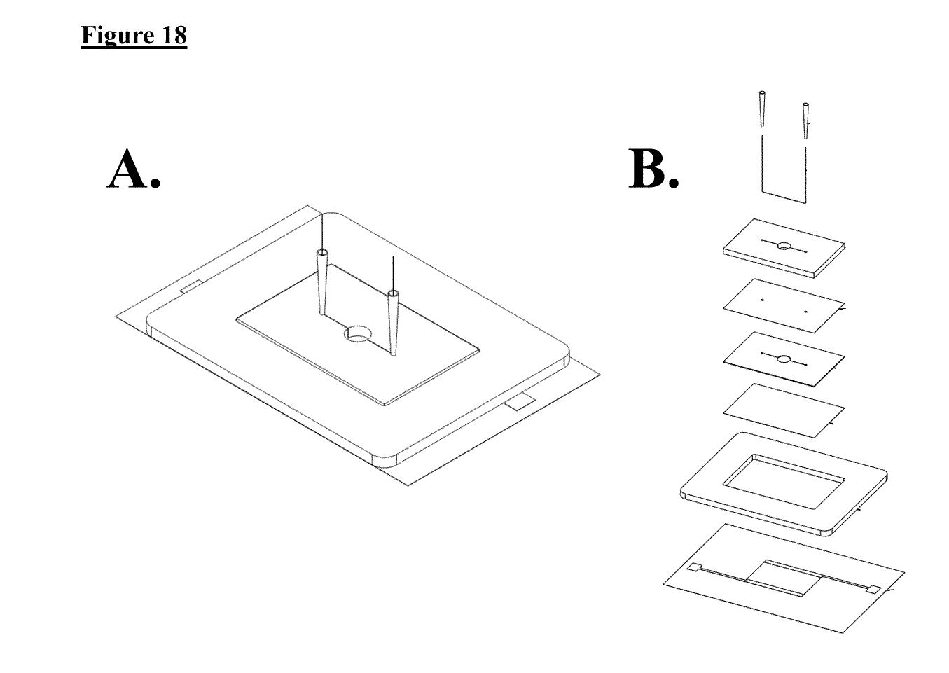

An assembly method, as shown in FIG. 18, utilizes standard printed circuit board techniques. The fluid paths and chambers are precut in each layer using a variety of methods (laser, die cutting). Each layer is assembled with standard techniques. The completed board can accept fluid by a variety of interconnects known in the art. Circuitry can easily be combined with fluid in this manner as some of the layers can have components or electric circuits in contact or insulated from the fluid. Some of these circuits can be ohmic heaters which can heat the sample, and the sample can cool adiabatically or through a forced means such as air or peltier effect.

As one example, assembly using this method also allows for easy lyophilization of reagents on chip. In a normal pc board construction the etched chambers have very small openings constrained to the height of each of the layers. This would make vapor transfer very slow and drying down the reagents impractical. In this construction the fluid pocket can be formed but not sealed by layers above. Reagents can be temperature stabilized on board and then the final layers can be added above the fluid chamber, thus sealing the temperature stabilized components on chip

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are photolithographic design, techniques for fabricating photolithographic materials, inclusion of cellular material, such as solidified liquid tissue integrated and detected with the various described components for analysis, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. An apparatus, comprising:
a support;
a cellular material mounted upon the support; and
a photomask comprising at least one non-blocking region and at least one blocking region, wherein the photomask is placed over the cellular material such that each of the at least one blocking region is positioned to correspond to a region of interest of the cellular material.

2. The apparatus of claim 1, wherein the support comprises a glass slide, a quartz slide or a transparent polymer slide.

3. The apparatus of claim 1, wherein the at least one blocking region comprises a metal.

4. The apparatus of claim 1, wherein the at least one blocking region comprises a polarizer.

5. The apparatus of claim 1, wherein the at least one blocking region is capable of limiting photon exposure.

6. The apparatus of claim 5, wherein the photons are derived from one or more of X-ray, UV, two-photon, or multi-photon illumination light.

7. The apparatus of claim 5 wherein the at least one non-blocking region is configured to allow passage of photons.

8. The apparatus of claim 1, further comprising a chambered microfluidic device comprising photosensitive material located above the cellular material, and wherein the photosensitive material comprises a plurality of access wells corresponding to a plurality of respective areas of interest of the cellular material.

9. The apparatus of claim 8, wherein the plurality of access wells are interconnected through a series of channels in the microfluidic device.

11. The apparatus of claim 8, wherein the chambered microfluidic device is configured for a coverage of 75%, 80%, 85%, 90% or more.

12. A method for selectively isolating cellular material, comprising:
positioning cellular material on a support;
depositing a photosensitive material on the cellular material;
applying a photomask comprising at least one non-blocking region and at least one blocking region onto the photosensitive material; and
exposing photons to the photosensitive material through the at least one non-blocking region in order to define at least one access well, and wherein each of the at least one blocking region corresponds to a region of interest of the cellular material.

13. The method of claim 12, wherein the photons are generated by arrays of micro- and nano-lasers light-emitting diodes, or photonic crystal devices, and/or reflected onto the sample by micro-mirror arrays.

14. A method to detect analytes, comprising:
solidifying liquid tissue;
positioning the solidified liquid tissue on a support;
depositing a photosensitive material on the solidified liquid tissue;
applying a photomask comprising at least one non-blocking region and at least one blocking region onto the photosensitive material;
exposing photons to the photosensitive material through the at least one non-blocking region in order to define at least one access well, and wherein each of the at least one blocking region corresponds to a region of interest of the solidified liquid tissue;
filling the at least one access well with a reaction mixture comprising agents and components necessary for reaction to detect analytes; and
performing the reaction simultaneously in the at least one access well, thereby detecting the analytes.

15. The method of claim 14, wherein the reaction comprises one or more reagents for PCR, real-time PCR, RT-PCR, flow cytometry, fluorescent labeling, FRET, DNA sequencing, protein-protein interaction assays, immunoassays, protein-nucleic acid assays.

16. The method of claim 14, wherein signal detection comprises scanning a completed reaction using a fluorescence scanner and/or a fluorescence microscope.

17. The method of claim 14, wherein depositing the solidified tissue and coverage of the at least one access well are each configured for detection of 10, 5, 2 or fewer cells per access well.

18. The method of claim 17, wherein the at least one access well are each substantially hexagonal.

19. The method of claim 14, performing the reaction simultaneously in the at least one access well is massively parallel.

20. The method of claim 14, wherein the liquid tissue is blood.
